(19) United States
(12) Patent Application Publication
(10) Pub. No.: US 2004/0266802 A1
(43) Pub. Date: Dec. 30, 2004

(54) Tricyclic analgesics

(76) Inventors: Alain Calvet, Ann Arbor, MI (US); Henri Jacobelli, Paray-Vieille-Poste (FR); Jocelyne Puaud, Montlhery (FR); Francois J. Roman, Vitry-Sur-Seine (FR); Jacques Hamon, Saint Maurice Montcouronne (FR); Agnes Grouhel, Meudon (FR)

Correspondence Address: WARNER-LAMBERT COMPANY, 2800 PLYMOUTH RD, ANN ARBOR, MI, 48105, US

(21) Appl. No.: 10/896,551

(22) Filed: Jul. 22, 2004

Related U.S. Application Data
(63) Continuation of application No. 09/980,965, filed on Dec. 4, 2001.
Application No. 09/980,965 is a 371 of international application No. PCT/EP00/05783, filed on Jun. 7, 2000.
(60) Provisional application No. 60/137,868, filed on Jun. 7, 1999.

(51) Int. Cl. C07D 471/10; A61K 31/4747

(52) U.S. Cl. 514/278; 546/15

(57) ABSTRACT

A tricyclic compound of Formula (I): wherein: R¹ is hydrogen or hydroxy; R² is hydrogen or hydroxy; or R¹ and R² together are oxygen; A is a bond, CH₂, CH CH₃, CH₂ CH₂ or C(CH₃)₂; R³ and R⁴ are the same or different and are hydrogen, halo, C₁-C₆ alkyl, C₁-C₄ alkoxy, trifluoromethyl NO₂, COR⁶, COOR⁶ or NR⁶R⁷, wherein R⁶ and R⁷ are the same or different and are hydrogen, C₁-C₆ alkyl or benzyl; R⁵ is hydrogen, C₁₋₆ alkyl, C₂₋₆ alkenyl, C₃₋₆ cycloalkyl, (O═C)—C₁₋₆ alkyl, (O═C)—C₂₋₆ alkenyl, (O═C)—C₃₋₆ cycloalkyl wherein said alkyl, alkenyl and cycloalkyl groups can be substituted by 1, 2 or 3 groups selected from halo, C₃-C₆ cycloalkyl, phenyl or substituted phenyl, and salts thereof, are particularly useful for treating, among other indications, neuropathic pain and other CNS disorders.

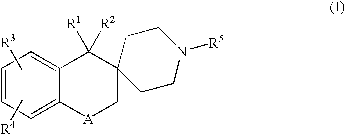

### FIELD OF THE INVENTION

The invention relates to organic compounds characterized as having a fused bicyclic ring system substituted with a spiro nitrogen-containing third ring. The compounds are effective for treating seizures and chronic pain in mammals.

### BACKGROUND OF THE INVENTION

Although chronic pain is a frequent condition in the population, its pathophysiology is not well understood. One possibility is that nociceptive spinal sensory neurons generate inappropriate activity after injury. Spinal sensory neurons become hyperexcitable and generate spontaneous impulses after injury in experimental animals, and in humans. Matzner and Devor (1992) proposed that the hyperexcitability associated with chronic pain results from an increase of Na channel density at the site of injury. It has also been hypothesized that, after nerve injury, changes in the kinetics and voltage-dependent characteristics of Na currents contribute to the ectopic impulse generation and hyperexcitability of spinal sensory neurons. Dorsal root ganglion (DRG) neurons possess a complex mix of Na currents, including a fast tetrodotoxin-sensitive (TTX-S) current and a slow TTX-resistant (TTX-R) current. In rat DRG neurons, PGE₂, adenosine and serotonin, three agents that produce hyperalgesia in vivo, increase the magnitude of the TTX-R current, and shift its conductance/voltage relationship in a hyperpolarized direction (Gold et al., 1996). Following nerve injury, TTX-R currents are down regulated in DRG neurons, and, in the same animals, TTX-S currents are upregulated (Cummings and Waxman, 1997). Using a Na channel specific antibody, Devor et al. (1993) evidenced an accumulation of Na channels in the neuroma resulting from a nerve section; the accumulation of Na channels at injured axonal tips may explain the ectopic channel excitability and the resulting pain and paresthesia which frequently complicate peripheral nerve injury in humans.

Injury to the axons of spinal sensory neurons appear to modify Na currents, substantially altering their excitability; Thus, selective blockers of Na channels can be used for the prevention or treatment of chronic pain in mammals. Sodium channel blockers have been shown effective in chronic pain syndromes, including trigeminal neuralgia, diabetic neuropathy, migraine prophylaxia and cancer pain (review by McQuay et al., 1995, British Medical Journal, 1995; 311: 1047-1052, and references cited therein).

However, pain due to acute or chronic nerve injury is difficult to treat, and is often resistant to conventional analgesics. Such compounds include some local anesthetics and anticonvulsants, for example lidocaine, etidocaine, benzocaine, tetracain, riluzole, phenytoin, and gabapentin. Most of them, even though such agents modulate Na channels, have limited clinical use because of high risks of adverse events. Lidocaine, for example, can cause cardiovascular collapse and resultant cardiac arrest. Benzocaine, can cause respiratory distress, as well as skin rash, erythema and oedema. The use of phenytoin for seizure disorders can result in hyperglycemia.

Because there is no effective chemical treatment for neuropathic pain, e.g. chronic pain, and since such pain is typically associated with diseases such as cancer, as well as severe physical injuries and diabetic neuropathy, the need continues to find compounds which can be utilized clinically without resulting in severe adverse events.

### SUMMARY OF THE INVENTION

The inventors have now discovered a series of tricyclic compounds which are potent antagonists of neuronal Na channels. The compounds are characterized as fused bicyclic ring systems having a spiro third ring substitution.

The invention therefore provides tricyclic compounds of Formula I:

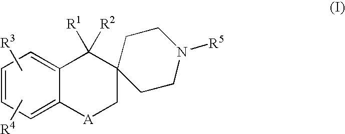

wherein:

R¹ is hydrogen or hydroxy;

R² is hydrogen or hydroxy; or

R¹ and R² together are oxygen;

A is a bond, CH₂, CH CH₃, CH₂ CH₂ or C(CH₃)₂;

R³ and R⁴ are the same or different and are hydrogen, halo, C₁-C₆ alkyl, C₁-C₄ alkoxy, trifluoromethyl, NO₂, COR⁶, COOR⁶ or NR⁶R⁷, wherein R⁶ and R⁷ are the same or different and are hydrogen, C₁-C₆ alkyl or benzyl;

R⁵ is hydrogen, C₁₋₆ alkyl, C₂₋₆ alkenyl, C₃₋₆ cycloalkyl, (O═C)—C₁₋₆ alkyl, (O═C)—C₂₋₆ alkenyl, (O═C)—C₃₋₆ cycloalkyl, wherein said alkyl, alkenyl and cycloalkyl groups can be substituted by 1, 2 or 3 groups selected from halo, C₃-C₆ cycloalkyl, phenyl or substituted phenyl, and the pharmacentically acceptable salts thereof.

The compounds of the invention are useful in the clinical management and treatment of various conditions such as seizure disorders, epilepsy, neuroprotection, preferably for conditions such as cerebral ischemia, hypoxia and head trauma, local anesthesia, pain, preferably acute, chronic, neuropathic, visceral and somatic pain, irritable bowel syndrom (IBS), the treatment of drug dependence, migraine and obsessional compulsive disorders.

Preferred compounds are those of Formula I wherein R⁵ is hydrogen, C₁-C₆ alkyl, optionally substituted with phenyl or a C₃-C₆ cycloalkyl group.

Other preferred compounds are those of Formula I wherein R⁵ is C₁-C₆ alkyl, optionally substituted with phenyl or a C₃-C₆ cycloalkyl group.

Other preferred compounds are those of Formula I wherein R⁵ is hydrogen, C₁-C₆ alkyl, optionally substituted with phenyl or a C₃-C₆ cycloalkyl group and R³ is hydrogen or C₁-C₄ alkoxy.

Most preferred compounds of the invention are compounds of Formula I wherein R¹ and R² together are oxygen and A is CH₂.

Other most preferred compounds are those of formula I wherein R⁵ is H, C₁-C₆ alkyl, optionally substituted with phenyl or a C₃-C₆ cycloalkyl group and R³ is hydrogen or C₁-C₄ alkoxy.

Another embodiment of this invention is a pharmaceutical formulation comprising a compound of Formula I admixed with a pharmaceutically acceptable carrier or diluent.

A further embodiment of the present invention is a method for treating a mammal suffering from pain and in need of treatment, comprising administering an effective amount of a compound of Formula I.

Still another embodiment of the invention is a method for treating a seizure disorder in a mammal in need of treatment, comprising administering a compound of Formula I.

Methods of treatment of all the further indications referred to above also fall within the scope of the present invention.

### DETAILED DESCRIPTION OF THE INVENTION

As used herein, the term “C₁-C₆ alkyl” means a straight or branched carbon chain made up of from one to six carbon atoms. Examples of C₁-C₆ alkyl groups include methyl, ethyl, isopropyl, sec-butyl, tert-butyl, isopentyl and n-hexyl.

“C₁-C₆ alkoxy” means the foregoing alkyl groups bonded through oxygen, for example methoxy, isopropoxy, and n-hexyloxy.

“C₂-C₆ alkenyl” means a straight or branched carbon chain having from two to six carbon atoms, with one carbon-carbon double bond present in the chain. Examples include ethenyl, 2-propenyl, 1-methyl-3-pentenyl, 1-ethyl-2-butenyl, and 5-hexenyl.

“C₃-C₆ cycloalkyl” means a non-aromatic cyclic ring having from three to six carbon atoms, examples being cyclopropyl, cyclobutyl, cyclopentyl and cyclohexyl.

The foregoing alkyl, alkenyl and cycloalkyl groups may be substituted by 1, 2 or 3 groups selected from halo, unsubstituted C₃-C₆ cycloalkyl, phenyl or substituted phenyl. “Halo” means chloro, bromo, fluoro and iodo. “Substituted phenyl” means a phenyl group having 1, 2 or 3 substituents selected from halo, hydroxy, nitro, unsubstituted C₁-C₆ alkyl, unsubstituted C₁-C₆ alkoxy, and NH₂.

Examples of C₁-C₆ alkyl groups substituted with cycloalkyl thus include cyclopropylmethyl, 1-cyclobutylethyl, 3-cyclohexylbutyl and 3,3-dicyclohexyl-propyl. Alkyl groups substituted with halo include chloromethyl, 1,2-dibromo-ethyl, trifluoromethyl, and 1-bromo-3-chloro-6-iodohexyl. Alkyl groups substituted with phenyl or with substituted phenyl include benzyl, 1-phenylpropyl, 1-methyl-3-phenyl-butyl, 3-chlorophenylmethyl, 2,3-dimethoxybenzyl, 3-(2-methyl-5-fluoro-6-nitrophenyl)-butyl, and 3,3-diphenylpropyl.

Examples of substituted C₂-C₆ alkenyl groups include 2-cyclobutylethenyl, 3-phenyl-2-butenyl, 1,1-dimethyl-3-chloro-3-butenyl, 4,4-diphenyl-3-butenyl, 2-(3-chlorophenyl)-3-cylclobutyl-4-hexenyl, and 1,2-difluoro-3-(2-phenyl-cyclobutyl)-4-pentenyl.

Examples of substituted C₃-C₆ cycloalkyl groups include 3-cyclo-pentylcyclohexyl, 2-phenylcyclobutyl, 3-chlorocyclopentyl, 2,2-dibromo-3-nitro-cyclohexyl, and 2,2-di-(3-methoxyphenyl)-cyclopropyl.

Examples of substituted C₁-C₆ alkoxy groups include trichloromethoxy, cyclopropylmethoxy, 1-methyl-2-phenylpropoxy and 2,3-di-(2,4-dinitrophenyl)-hexyloxy.

The alkyl, alkenyl and cycloalkyl substituent groups can be bonded through a carbonyl (O═C) group. Examples include acetyl, pivaloyl, 1-oxo-3-pentenyl, 1-oxocyclobutylmethyl, 1-oxo-3-phenyl-4-cyclohexylpentyl, and 1-oxo-(3-phenylcyclopentyl)-methyl.

“-A-” in Formula I is defined as a bond, —CH₂—, —CH—CH₃, —CH—(CH₃)₂ and —CH₂CH₂—; the invention compounds can thus have the following general structures:

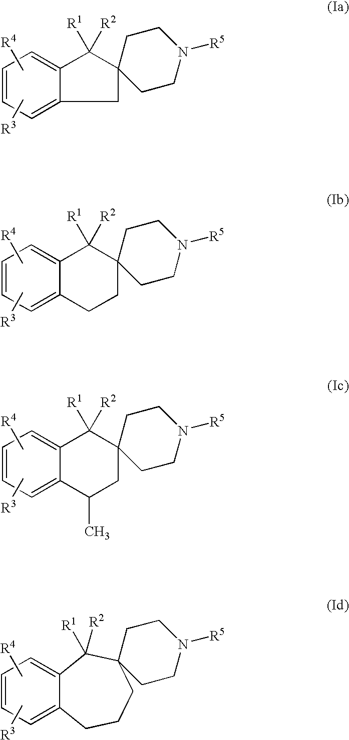

Preferred compounds of the present invention include the following:

3,4-Dihydro-1-oxospiro[naphthalene-2(1H),4′-piperidine];

1′-cyclopropylmethyl-3,4-Dihydro-1-oxospiro[naphthalene-2(1H),4′-piperidine];

1′-cyclobutylmethyl-3,4-Dihydro-1-oxospiro[naphthalene-2(1H),4′-piperidine];

1′-cyclohexylmethyl-3,4-Dihydro-1-oxospiro[naphthalene-2(1H),4′-piperidine];

1′-phenylethyl-3,4-Dihydro-1-oxospiro[naphthalene-2(1H),4′-piperidine];

1′-cyclopropylethyl-3,4-Dihydro-1-oxospiro[naphthalene-2(1H),4′-piperidine];

1′-cinnamyl-3,4-Dihydro-1-oxospiro[naphthalene-2(1H),4′-piperidine];

1′-(3,3-diphenylpropyl)-3,4-Dihydro-1-oxospiro[naphthalene-2(1H),4′-piperidine];

1′-(cyclopropylmethyl)-3,4-Dihydro-5,7-dimethyl-1-oxospiro[naphthalene-2(1H),4′-piperidine];

1′-(cyclopropylmethyl)-3,4-Dihydro-6-methoxy-1-oxospiro[naphthalene-2(1H),4′-piperidine];

1′-(cyclopropylmethyl)-3,4-Dihydro-5-methoxy-1-oxospiro[naphthalene-2(1H),4′-piperidine];

1′-(cyclopropylmethyl)-3,4-Dihydro-7-methoxy-1-oxospiro[naphthalene-2(1H),4′-piperidine];

1′-(cyclopropylmethyl)-3,4-Dihydro-7-nitro-1-oxospiro[naphthalene-2(1H),4′-piperidine];

1′-(cyclopropylmethyl)-7-Amino-3,4-dihydro-1-oxospiro[naphthalene-2(1H),4′-piperidine];

1′-(cyclopropylmethyl)-7-Chloro-3,4-dihydro-1-oxospiro[naphthalene-2(1H),4′-piperidine];

3,4-dihydro-1′-methyl-1-oxospiro(naphthalene-2(1H),4′-piperidine);

1′-allyl-3,4-dihydro-1-oxospiro(naphthalene-2(1H),4′-piperidine);

3,4-dihydro-1′-(2-methylpropyl)-1-oxospiro(naphthalene-2(1H),4′-piperidine);

1′-cyclopropionyl-3,4-dihydro-1-oxospiro(naphthalene-2(1H),4′-piperidine);

3,4-dihydro-1-oxospiro(naphthalene-2(1H),4′-piperidine)-1′(trans-2-phenyl-methylcyclopropyl);

3,4-dihydro-1′-benzyl-1-oxospiro(naphthalene-2(1H),4′-piperidine);

3,4-dihydro-1′-(di-p-fluorobenzhydryl)-1-oxospiro(naphthalene-2(1H),4′-piperidine);

1′-cyclopropylmethyl-1,3,4-trihydro-1-hydroxy-spiro(naphthalene-2(1H),4′-piperidine);

1′-cyclopropylmethyl-1,2,3,4-tetrahydro-spiro(naphthalene-2(1H),4′-piperidine);

1′-cyclopropylmethyl-1,3-dihydro-1-oxospiro-[2H-indene-2,4′-piperidine];

1′-(cyclopropylmethyl)-8,9-dihydrospiro[6H-benzocycloheptene-6,4′-piperidin]-5(7H)-one;

1′-cyclopropylmethyl-3,4-dihydro-4-methyl-1-oxospiro(naphthalene-2(1H),4′-piperidine);

6-chloro-1′-cyclopropylmethyl-3,4-dihydro-1-oxospiro(naphthalene-2(1H),4′-piperidine);

1′-cyclopropylmethyl-3,4-dihydro-6-fluoro-1-oxospiro(naphthalene-2(1H),4′-piperidine);

1′-cyclopropylmethyl-3,4-dihydro-6,7-dimethoxy-1-oxospiro(naphthalene-2(1H),4′-piperidine);

1′-(1-cyclopropyl-1-ethyl)-3,4-dihydro-1-oxospiro(naphthalene-2(1H),4′-piperidine);

1′-(3-pentene)-3,4-dihydro-1-oxospiro(naphthalene-2(1H),4′-piperidine;

1′-(3-phenylpropyl)-3,4-dihydro-1-oxospiro(naphthalene-2(1H),4′-piperidine;

1′-cinnamyl-3,4-dihydro-6-methoxy-1-oxospiro(naphthalene-2(1H),4′-piperidine;

1′-cyclopropylethyl-3,4-dihydro-5-methoxy-1-oxospiro(naphthalene-2(1H),4′-piperidine;

1′-cyclobutylmethyl-3,4-dihydro-5-methoxy-1-oxospiro(naphthalene-2(1H),4′-piperidine;

1′-cyclohexylmethyl-3,4-dihydro-5-methoxy-1-oxospiro(naphthalene-2(1H),4′-piperidine;

1′-cinnamyl-3,4-dihydro-5-methoxy-1-oxospiro(naphthalene-2(1H),4′-piperidine;

1′-(2-phenylethyl)-3,4-dihydro-5-methoxy-1-oxospiro(naphthalene-2(1H),4′-piperidine;

1′-(3-phenylpropyl)-3,4-dihydro-5-methoxy-1-oxospiro(naphthalene-2(1H),4′-piperidine;

1′-(3,3′diphenylpropyl)-3,4-dihydro-5-methoxy-1-oxospiro(naphthalene-2(1H),4′-piperidine;

1′-cyclopropylethyl-3,4-dihydro-6-methoxy-1-oxospiro(naphthalene-2(1H),4′-piperidine;

1′-cyclobutylmethyl-3,4-dihydro-6-methoxy-1-oxospiro(naphthalene-2(1H),4′-piperidine;

1′-cyclohexylmethyl-3,4-dihydro-6-methoxy-1-oxospiro(naphthalene-2(1H),4′-piperidine;

1′-cinnamyl-3,4-dihydro-6-methoxy-1-oxospiro(naphthalene-2(1H),4′-piperidine;

1′-(2-phenylethyl)-3,4-dihydro-6-methoxy-1-oxospiro(naphthalene-2(1H),4′-piperidine;

1′-(3-phenylpropyl)-3,4-dihydro-6-methoxy-1-oxospiro(naphthalene-2(1H),4′-piperidine;

1′-(3,3′diphenylpropyl)-3,4-dihydro-6-methoxy-1-oxospiro(naphthalene-2(1H),4′-piperidine;

1′-cyclopropylethyl-3,4-dihydro-7-methoxy-1-oxospiro(naphthalene-2(1H),4′-piperidine;

1′-cyclobutylmethyl-3,4-dihydro-7-methoxy-1-oxospiro(naphthalene-2(1H),4′-piperidine;

1′-cyclohexylmethyl-3,4-dihydro-7-methoxy-1-oxospiro(naphthalene-2(1H),4′-piperidine;

1′-cinnamyl-3,4-dihydro-7-methoxy-1-oxospiro(naphthalene-2(1H),4′-piperidine;

1′-(2-phenylethyl)-3,4-dihydro-7-methoxy-1-oxospiro(naphthalene-2(1H),4′-piperidine;

1′-(3-phenylpropyl)-3,4-dihydro-7-methoxy-1-oxospiro(naphthalene-2(1H),4′-piperidine;

1′-(3,3′diphenylpropyl)-3,4-dihydro-7-methoxy-1-oxospiro(naphthalene-2(1H),4′-piperidine;

1′-cyclopropylethyl-3,4-dihydro-4-methyl-1-oxospiro(naphthalene-2(1H),4′-piperidine;

1′-cyclobutylmethyl-3,4-dihydro-4-methyl-1-oxospiro(naphthalene-2(1H),4′-piperidine;

1′-cyclohexylmethyl-3,4-dihydro-4-methyl-1-oxospiro(naphthalene-2(1H),4′-piperidine;

1′-cinnamyl-3,4-dihydro-4-methyl-1-oxospiro(naphthalene-2(1H),4′-piperidine;

1′-(2-phenylethyl)-3,4-dihydro-4-methyl-1-oxospiro(naphthalene-2(1H),4′-piperidine;

1′-(3-phenylpropyl)-3,4-dihydro-4-methyl-1-oxospiro(naphthalene-2(1H),4′-piperidine;

1′-(3,3′diphenylpropyl)-3,4-dihydro-4-methyl-1-oxospiro(naphthalene-2(1H),4′-piperidine;

6-chloro-1′-cyclopropylethyl-3,4-dihydro-1-oxospiro(naphthalene-2(1H),4′-piperidine;

6-chloro-1′-cyclobutylmethyl-3,4-dihydro-1-oxospiro(naphthalene-2(1H),4′-piperidine;

6-chloro-1′-cyclohexylmethyl-3,4-dihydro-1-oxospiro(naphthalene-2(1H),4′-piperidine;

6-chloro-1′-cinnamyl-3,4-dihydro-1-oxospiro(naphthalene-2(1H),4′-piperidine;

6-chloro-1′-(2-phenylethyl)-3,4-dihydro-1-oxospiro(naphthalene-2(1H),4′-piperidine;

6-chloro-1′-(3-phenylpropyl)-3,4-dihydro-1-oxospiro(naphthalene-2(1H),4′-piperidine; and

6-chloro-1′-(3,3′diphenylpropyl)-3,4-dihydro-1-oxospiro(naphthalene-2(1H),4′-piperidine.

3,4-Dihydro-6-methoxy-1-oxospiro[naphthalene-2(1H),4′-piperidine]

More preferred compounds of the invention include the following:

1′-cyclopropylmethyl-3,4-Dihydro-1-oxospiro[naphthalene-2(1H),4′-piperidine];

1′-cyclopropylethyl-3,4-Dihydro-1-oxospiro[naphthalene-2(1H),4′-piperidine];

1′-cinnamyl-3,4-Dihydro-1-oxospiro[naphthalene-2(1H),4′-piperidine];

1′-(3,3-diphenylpropyl)-3,4-Dihydro-1-oxospiro[naphthalene-2(1H),4′-piperidine];

1′-(cyclopropylmethyl)-3,4-Dihydro-5,7-dimethyl-1-oxospiro[naphthalene-2(1H),4′-piperidine];

1′-(cyclopropylmethyl)-3,4-Dihydro-6-methoxy-1-oxospiro[naphthalene-2(1H),4′-piperidine];

1′-(cyclopropylmethyl)-3,4-Dihydro-5-methoxy-1-oxospiro(naphthalene-2(1H),4′-piperidine);

1′-(cyclopropylmethyl)-3,4-Dihydro-7-methoxy-1-oxospiro[naphthalene-2(1H),4′-piperidine];

1′-(cyclopropylmethyl)-3,4-Dihydro-7-nitro-1-oxospiro[naphthalene-2(1H),4′-piperidine];

1′-(cyclopropylmethyl)-7-Amino-3,4-dihydro-1-oxospiro[naphthalene-2(1H),4′-piperidine];

1′-(cyclopropylmethyl)-7-Chloro-3,4-dihydro-1-oxospiro[naphthalene-2(1H),4′-piperidine];

1′-cyclopropylmethyl-1,3,4-trihydro-1-hydroxy-spiro(naphthalene-2(1H),4′-piperidine);

1′-cyclopropylmethyl-1,2,3,4-tetrahydro-spiro(naphthalene-2(1H),4′-piperidine);

1′-cyclopropylmethyl-1,3-dihydro-1-oxospiro-[2H-indene-2,4′-piperidine];

1′-(cyclopropylmethyl)-8,9-dihydrospiro[6H-benzocycloheptene-6,4′-piperidin]-5(7H)-one;

1′-cyclopropylmethyl-3,4-dihydro-4-methyl-1-oxospiro(naphthalene-2(1H),4′-piperidine);

6-chloro-1′-cyclopropylmethyl-3,4-dihydro-1-oxospiro(naphthalene-2(1H),4′-piperidine);

1′-cyclopropylmethyl-3,4-dihydro-6-fluoro-1-oxospiro(naphthalene-2(1H),4′-piperidine);

1′-cyclopropylmethyl-3,4-dihydro-6,7-dimethoxy-1-oxospiro(naphthalene-2(1H),4′-piperidine);

1′-(1-cyclopropyl-1-ethyl)-3,4-dihydro-1-oxospiro(naphthalene-2(1H),4′-piperidine);

1′-(3-pentene)-3,4-dihydro-1-oxospiro(naphthalene-2(1H),4′-piperidine;

1′-(3-phenylpropyl)-3,4-dihydro-1-oxospiro(naphthalene-2(1H),4′-piperidine;

1′-cinnamyl-3,4-dihydro-6-methoxy-1-oxospiro(naphthalene-2(1H),4′-piperidine;

1′-cyclopropylethyl-3,4-dihydro-5-methoxy-1-oxospiro(naphthalene-2(1H),4′-piperidine;

1′-cyclobutylmethyl-3,4-dihydro-5-methoxy-1-oxospiro(naphthalene-2(1H),4′-piperidine;

1′-cinnamyl-3,4-dihydro-5-methoxy-1-oxospiro(naphthalene-2(1H),4′-piperidine;

1′-(3-phenylpropyl)-3,4-dihydro-5-methoxy-1-oxospiro(naphthalene-2(1H),4′-piperidine;

1′-(3,3′diphenylpropyl)-3,4-dihydro-5-methoxy-1-oxospiro(naphthalene-2(1H),4′-piperidine;

1′-cyclopropylethyl-3,4-dihydro-6-methoxy-1-oxospiro(naphthalene-2(1H),4′-piperidine;

1′-cyclobutylmethyl-3,4-dihydro-6-methoxy-1-oxospiro(naphthalene-2(1H),4′-piperidine;

1′-cinnamyl-3,4-dihydro-6-methoxy-1-oxospiro(naphthalene-2(1H),4′-piperidine;

1′-(3-phenylpropyl)-3,4-dihydro-6-methoxy-1-oxospiro(naphthalene-2(1H),4′-piperidine;

1′-(3,3′diphenylpropyl)-3,4-dihydro-6-methoxy-1-oxospiro(naphthalene-2(1H),4′-piperidine;

1′-cyclopropylethyl-3,4-dihydro-7-methoxy-1-oxospiro(naphthalene-2(1H),4′-piperidine;

1′-cinnamyl-3,4-dihydro-7-methoxy-1-oxospiro(naphthalene-2(1H),4′-piperidine;

1′-(3-phenylpropyl)-3,4-dihydro-7-methoxy-1-oxospiro(naphthalene-2(1H),4′-piperidine;

1′-(3,3′diphenylpropyl)-3,4-dihydro-7-methoxy-1-oxospiro(naphthalene-2(1H),4′-piperidine;

1′-cyclopropylethyl-3,4-dihydro-4-methyl-1-oxospiro(naphthalene-2(1H),4′-piperidine;

1′-cyclobutylmethyl-3,4-dihydro-4-methyl-1-oxospiro(naphthalene-2(1H),4′-piperidine;

1′-cinnamyl-3,4-dihydro-4-methyl-1-oxospiro(naphthalene-2(1H),4′-piperidine;

1′-(3-phenylpropyl)-3,4-dihydro-4-methyl-1-oxospiro(naphthalene-2(1H),4′-piperidine;

1′-(3,3′diphenylpropyl)-3,4-dihydro-4-methyl-1-oxospiro(naphthalene-2(1H),4′-piperidine;

6-chloro-1′-cyclopropylethyl-3,4-dihydro-1-oxospiro(naphthalene-2(1H),4′-piperidine;

6-chloro-1′-cinnamyl-3,4-dihydro-1-oxospiro(naphthalene-2(1H),4′-piperidine;

6-chloro-1′-(3-phenylpropyl)-3,4-dihydro-1-oxospiro(naphthalene-2(1H),4′-piperidine; and

6-chloro-1′-(3,3′diphenylpropyl)-3,4-dihydro-1-oxospiro(naphthalene-2(1H),4′-piperidine.

3,4-Dihydro-6-methoxy-1-oxospiro[naphthalene-2(1H),4′-piperidine]

Most preferred compounds of the invention include the following:

1′-cyclopropylmethyl-3,4-Dihydro-1-oxospiro[naphthalene-2(1H),4′-piperidine];

1′-cyclopropylethyl-3,4-Dihydro-1-oxospiro[naphthalene-2(1H),4′-piperidine];

1′-cinnamyl-3,4-Dihydro-1-oxospiro[naphthalene-2(1H),4′-piperidine];

1′-(3,3-diphenylpropyl)-3,4-Dihydro-1-oxospiro[naphthalene-2(1H),4′-piperidine];

1′-(cyclopropylmethyl)-3,4-Dihydro-6-methoxy-1-oxospiro[naphthalene-2(1H),4′-piperidine];

1′-(3-phenylpropyl)-3,4-dihydro-1-oxospiro(naphthalene-2(1H),4′-piperidine;

1′-cyclobutylmethyl-3,4-dihydro-5-methoxy-1-oxospiro(naphthalene-2(1H),4′-piperidine;

1′-cyclopropylethyl-3,4-dihydro-6-methoxy-1-oxospiro(naphthalene-2(1H),4′-piperidine;

1′-cyclobutylmethyl-3,4-dihydro-6-methoxy-1-oxospiro(naphthalene-2(1H),4′-piperidine;

1′-(3-phenylpropyl)-3,4-dihydro-6-methoxy-1-oxospiro(naphthalene-2(1H),4′-piperidine;

1′-cyclopropylethyl-3,4-dihydro-7-methoxy-1-oxospiro(naphthalene-2(1H),4′-piperidine;

3,4-Dihydro-6-methoxy-1-oxospiro[naphthalene-2(1H),4′-piperidine]

The compounds of Formula I are characterized by being bicyclic rings having a spiro ring as a substituent group. The spiro ring contains a nitrogen atom (i.e. N—R⁵), which can be basic in nature when R⁵ is a group such as alkyl, alkenyl or cycloalkyl. Such basic compounds readily form pharmaceutically acceptable salts by reaction with common inorganic and organic acids. Typical acids utilized to form the pharmaceutically acceptable salts of the invention include hydro-chloric, sulfuric, sulfamic, phosphoric, citric, succinic, glutamic, maleic, lactic, tartaric, p-toluenesulfonic, benzoic, oxalic and salicyclic acid. The salts are prepared by simply contacting the Spiro base with the appropriate acid, generally in a solvent such as methanol or diethyl ether. The salts generally are highly crystalline, readily precipitate, and are recovered by filtration. They can be further purified if desired by recrystallization from common solvents such as methanol, ethyl acetate, acetone and tetrahydrofuran.

The invention compounds of Formula I are readily prepared by methodologies well known in the art of organic chemistry. It is preferred to simply first prepare a compound wherein R⁵ is hydrogen, and then react this compound with R⁵-alkylating or-acylating agents. Such reaction is shown in scheme I below:

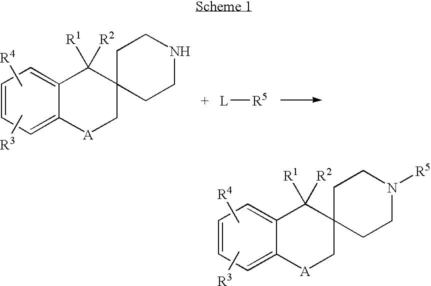

where R¹, R², R³, R⁴, and A have the meanings given above, and “L” is a normal leaving group (e.g. halo such as chloro or bromo, or a silyl derivative such as trimethylsilyl).

The foregoing reaction is carried out by combining approximately equimolar quantities of the spiro amine with the alkylating or acylating agent (i.e. L-R⁵), generally in an unreactive organic solvent such as tetrahydrofuran, dimethylsulfoxide, or N,N-dimethylformamide. A base such as triethylamine or NaHCO₃ can be utilized to act as an acid scavenger if desired. The reaction typically is substantially complete after about 2-20 h, when conducted at a temperature is about 25° C. to about 60° C. The product is readily isolated by removing the reaction solvent, and further purification can be achieved if desired by normal means such as salt formation, crystallization, and chromatography.

The required starting material, i.e. the spiro amine, can be synthesized from readily available reactants, utilizing any of several methods:

In one method, an N-protected form of a derivatized diethylamine is reacted with a bicyclic ketone according to scheme 2:

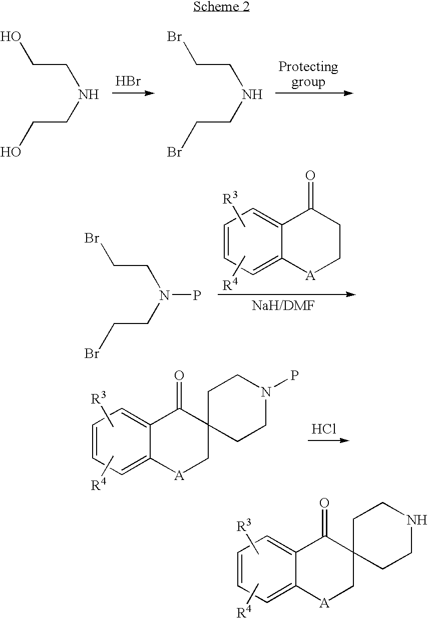

In the above scheme 2, P is an amine-protecting group that is easily removed, for example ethoxycarbonyl or benzyl. The protected diethylamine derivative is readily reacted with a bicyclic ketone in the presence of a strong base such as NaH. This reaction results in formation of the spiro amino derivative, which is readily de-protected by conventional means, for instance by reaction with hydrochloric acid.

Scheme 2 illustrates the preparation of keto substituted starting materials, i.e. where R¹ and R² together are oxo. Such compounds are easily converted to the corresponding alcohol (R¹ is H, R² is OH) by reaction with a reducing agent such as NaBH₄, generally in a solvent such as methanol or ethanol.

The alcohol can be further reduced by catalytic hydrogenation, for example by reaction with hydrogen gas in the presence of 10% palladium on carbon. These reactions are illustrated in Scheme 3:

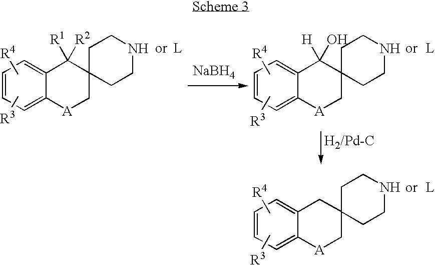

The invention compounds of Formula I can alternatively be prepared by starting with a suitably substituted piperidine derivative, as shown in Scheme 4:

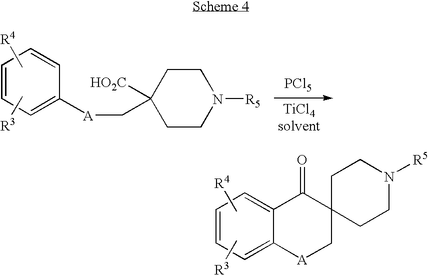

The cyclization reaction is accomplished by reacting the substituted piperidine with strong dehydrating agents such as phosphorus pentachloride and titanium tetrachloride, generally in an unreactive organic solvent such as benzene, toluene, xylene, or chloroform. The reaction normally is complete within 2 h when carried out at a temperature of 30° C. to 60° C.

The cyclized product is a compound of Formula I wherein R¹ and R² together are oxo, which, as noted above in Scheme 3, can be reduced to the corresponding alcohol or alkane (R¹ and R² both hydrogen).

The substituted piperidine required for the above reaction is readily prepared as shown in Scheme 5:

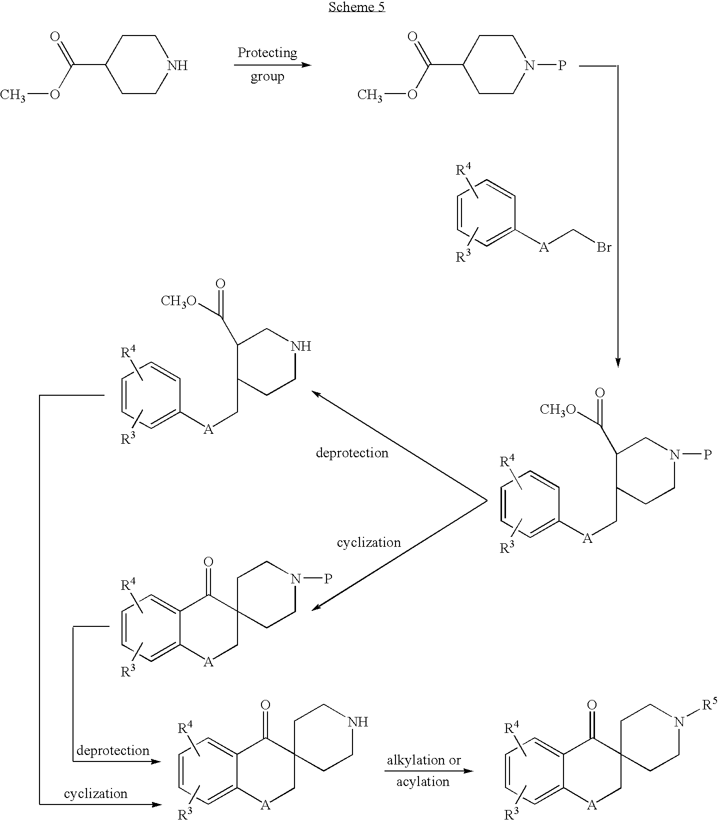

In Scheme 5, the methyl 4-piperidine formate is reacted with an amine-protecting agent (i.e. to insert P). Typical amine-protecting groups include tert-butoxy carbonyl, benzyl and trimethylsilyl. The protected piperidine derivative is next reacted with a phenyl alkyl halide, for example phenylethyl bromide (where A is CH₂) or 2-phenylpropyl iodide (where A is CH—CH₃) or 3-phenylpropyl iodide (where A is CH₂ CH₂), in the presence of a strong base such as NaH or lithium diisopropylamide (LDA), generally in an unreactive solvent such as tetrahydro-furan, or benzene. The reaction, carried out at about −20° C., generally is substantially complete after about 2-4 h. The alkylated piperidine can then be deprotected (removal of the L-protecting group) and cyclized by reaction with PCl₅ and TiCl₄, or it can be cyclized first, and the L-protecting group subsequently removed.

The following detailed examples illustrate the synthesis of specific compounds provided by this invention. The examples are representative only, and are not intended to be limiting in any respect.

### EXAMPLE 1

### 3,4-Dihydro-1-oxospiro[naphthalene-2(1H),4′-piperidine]

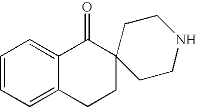

Stage 1: Bis(2-bromoethyl)amine Hydrobromide.

157.5 g (1.5 mol) of diethanolamine and then, with stirring, 1.35 l of 48% HBr (exothermic reaction) are introduced into a 2 l three-necked flask which can be equipped either for reflux or for distillation. The solution is heated at a bath temperature of 180-200° C., in order to distill off a volume of 350 ml at a vapour temperature of 122° C. The device is adjusted to the reflux position and is maintained for 1 h. A further distillation is carried out as above, in order to collect a distillate of 465 ml. The device is again adjusted for reflux for 3.75 h and then 400 ml are distilled off. The mixture is cooled and 300 ml of ethyl acetate are added to the residue. The suspension is stirred for 1 h in an ice bath. The precipitate is filtered off and then washed with ethyl acetate. 367 g of a white crystalline product are obtained. Yd=78.5%, M.p. (° C.)=130-135° C.

Stage 2: Ethyl bis(2-bromoethyl)carbamate.

367 g (1.17 mol) of the product obtained in the preceding stage and then 108 ml, i.e. 122.6 g (1.13 mol), of ethyl chloroformate are added, with stirring, to a 4 l reactor containing 1.8 l of a water/ice mixture. Approximately 1.3 l of a 2N sodium hydroxide solution are run into the solution over 5 min in order to achieve a continuing pH of 11, while maintaining a temperature below 5° C. The mixture is stirred for 5 min and then acidified to pH 1 with concentrated HCl. Extraction is carried out with 3 times 1 l of ethyl ether. The organic phase is washed with 3 times 500 ml of demineralized water and then dried over Na₂SO₄. The solvent is evaporated. The residue is chromatographed by eluting with CH₂Cl₂. 208.5 g of product are obtained. Yd=58%, TLC (CH₂Cl₂): R_{f}=0.6,

N.M.R.: CDCl₃ ¹H ((ppm): 1.2 (t, 3H), 3.4-3.55 (m, 4H), 3.6-3.7 (m, 4H), 4.1-4.2 (q, 2H)).

Stage 3: Ethyl 3,4-dihydro-1-oxospiro [naphthalene-2(1H),4′-piperidine]-1′-carboxylate.

69 g (0.472 mol) of 1-tetralone and 234 ml of DMF, dried beforehand over molecular sieve, are introduced into a reactor which is protected from moisture and which is under an inert atmosphere. The solution is cooled to −15° C. with a dry ice/acetone bath and 34.6 g (1.15 mol) of 80% sodium hydride, as a dispersion in mineral oil, are added thereto. The temperature is allowed to rise to approximately 20-25° C. (exothermic reaction). The reaction mixture is stirred for 1.5 h at a temperature below 30° C.

At the same time, a solution of 208 g (0.69 mol) of ethyl bis(2-bromoethyl)carbamate in 234 ml of DMF (dried beforehand over molecular sieve) is cooled, in a reactor which is protected from moisture and which is under an inert atmosphere, to −25° C. with a dry ice/acetone bath. The reaction liquors, prepared at the same time, are introduced by transfer under nitrogen and run in over 10 min at a temperature of −25° C. The temperature is allowed to rise (exothermic reaction with rise in the temperature to 45° C.). The reaction mixture is then cooled in order to maintain it at approximately 30° C. It is subsequently brought to 50° C. for 2 h and then the solvent is evaporated at 50° C. under a vacuum of less than 1 mm Hg. The residue is taken up in 1.2 l of ice-cold water and extracted with 3 times 600 ml of ether. The organic phase is washed with 3 times 500 ml of demineralized water and then dried over Na₂SO₄. After evaporating the solvent, a dark brown oily residue is obtained which is purified by fast chromatography by eluting with CH₂Cl₂ gradually enriched with acetone. 57.7 g (0.2 mol) of product are obtained (Yd=42.5%), TLC (97/3 CH₂Cl₂/acetone): R_{f}=0.45.

N.M.R.: CDCl₃ ¹H ((ppm): 1.15 (t, 3H), 1.4 (m, 2H), 1.8-2.0 (m, 4H), 2.85-2.95 (m, 2H), 3.45-3.55 (m, 4H), 4.0-4.1 (q, 2H), 7.1 (d, 1H), 7.2 (dd, 1H), 7.35 (dd, 1H), 7.9 (d, 1H)).

Stage 4: 3,4-Dihydro-1-oxospiro[naphthalene-2(1H),4′-piperidine).

57.7 g (0.2 mol) of ethyl 3,4-dihydro-1-oxospiro-[naphthalene-2(1H),4′-piperidine]-1′-carboxylate and then 1.6 l of 6N HCl are introduced into a reactor. The mixture is stirred and brought to reflux for 14 h. It is then cooled and extracted with twice 500 ml of ethyl ether. The aqueous phase is basified while cold with NaOH and extracted with 3 times 500 ml of ethyl ether. The organic phase is washed and dried over Na₂SO₄. After evaporating the solvent, the residue is purified by chromatography by eluting with CH₂Cl₂ gradually enriched with methanol containing 10% NH₄OH.

Weight: 31 g, Yd=72%, TLC (90/10 CH₂Cl₂/MeOH containing 10% NH₄OH): R_{f}=0.2-0.35. N.M.R.: CDCl₃ ¹H ((ppm) Base: 1.35-1.45 (m, 2H), 1.8-1.9 (m, 2H), 2.0 (t, 2H), 2.1 (s, 1H), 2.75-2.85 (m, 2H), 2.85-3.0 (m, 4H), 7.1 (d, 1H), 7.2 (dd, 1H), 7.35 (dd, 1H), 7.9 (d, 1H)).

The hydrochloride is prepared by addition of approximately 5N ethereal hydrochloric acid to a solution of the product in CH₂Cl₂. The mixture is concentrated to dryness and then the product is crystallized from a methanol/ether mixture.

White powder, M.p.=235° C., TLC (90/10 CH₂Cl₂/MeOH containing 10% NH₄OH): R_{f}=0.35. Analysis conforms to C₁₄H₁₈ClNO. IR: 2995, 2700, 1675, 1600, 1440, 1395, 1210, 1090, 990, 750, 740 cm⁻¹

### EXAMPLE 2

### 1′-cyclopropylmethyl-3,4-Dihydro-1-oxospiro[naphthalene-2(1H),4′-piperidine]

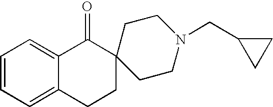

10 g (46.4 mmol) of 3,4-dihydro-1-oxospiro[naphthalene-2(1H),4′-piperidine] and then 80 ml of DMF and 36.2 ml of THF are introduced into a three-necked flask. 6.89 g (51 mmol) of (bromomethyl)cyclopropane and 7.8 g (92.8 mmol) of NaHCO₃ are added. The suspension is brought to reflux and then maintained for 1.5 h. The solvents are evaporated at 50° C. under a vacuum of less than 1 mm Hg. The residue is taken up in 200 ml of water and extracted with 3 times 100 ml of ether. The ethereal phase is extracted with 100 ml of 1N HCl and then twice with 50 ml of water. The aqueous phase is basified while cold with concentrated NaOH and extracted 3 times with 100 ml of ether. The organic phase is washed with an NaCl solution and dried over Na₂SO₄. Once the solvent has been evaporated, 12 g of an oily residue are obtained. The hydrochloride is prepared by addition of approximately 5N ethereal hydrochloric acid to a solution of the crude product in CH₂Cl₂. The mixture is concentrated to dryness and then the product is crystallized by addition of 20 ml of ether to a methanolic solution of the product, crystallization is allowed to take place overnight at 20-25° C. and then the product is filtered off and washed with water. After drying, 8.4 g of product are obtained.

White powder, M.p.=243° C., TLC (95/5 CH₂Cl₂/MeOH containing 10% NH₄OH): R_{f}=0.55. Analysis conforms to C₁₈H₂₄ClNO. N.M.R.: CDCl₃ ¹H ((ppm) HCl: 0.4-0.55 (m, 2H), 0.75-0.8 (m, 2H), 1.3-1.4 (m, 1H), 2.1-2.2 (m, 4H), 2.4-2.55 (m, 2H), 2.85-2.9 (m, 2H), 3.0-3.05 (m, 2H), 3.2-3.3 (m, 2H), 3.5-3.6 (m, 2H), 7.2-7.25 (m, 1H), 7.3-7.35 (m, 1H), 7.5-7.6 (m, 1H), 7.95-8.0 (m, 1H), 12.0-12.2 (m, 1H). IR: 2990, 2700, 1675, 1600, 1420, 1395, 1320, 1080, 980, 900, 760, 740 cm⁻¹

The corresponding iodomethylate was also obtained. MP: 162° C.

### EXAMPLE 3

### 1′-cyclobutylmethyl-3,4-Dihydro-1-oxospiro[naphthalene-2(1H),4′-piperidine]

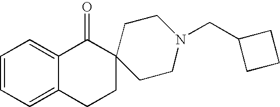

The method described for Example 2, using cyclobutylmethyl bromide, results in the product in the hydrochloride form. Beige powder, M.p.=235° C.

TLC: (92/8 CH₂Cl₂/MeOH containing 10% NH₄OH): R_{f}=0.7. Analysis conforms to C₁₉H₂₆ClNO. N.M.R.: CDCl₃ ¹H ((ppm) HCl: 1.8-2.25 (m, 10H), 2.4-2.5 (m, 2H), 2.95-3.1 (m, 5H), 3.1-3.25 (m, 2H), 3.25-3.35 (m, 2H), 7.2-7.35 (m, 2H), 7.45-7.55 (m, 1H), 7.9-8.0 (m, 1H), 11.95-12.15 (m, 1H)) IR: 3400, 2900, 2650, 2500, 1680, 1590, 1430, 1360, 1300, 1220, 1140, 1100, 1040, 960, 930, 900, 800, 770, 740, 640 cm⁻¹

### EXAMPLE 4

### 1′-cyclohexylmethyl-3,4-Dihydro-1-oxospiro[naphthalene-2(1H),4′-piperidine]

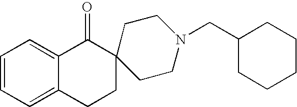

The method described for Example 2, using cyclohexylmethyl bromide, results in the product in the hydrochloride form. Beige powder, M.p.=265° C.

TLC: (93/7 CH₂Cl₂/MeOH containing 10% NH₄OH): R_{f}=0.8. Analysis conforms to C₂₁H₃₀ClNO. N.M.R.: CDCl₃ ¹H ((ppm) HCl: 0.95-1.3 (m, 5H), 1.6-2.1 (m, 10H), 2.5-2.65 (m, 2H), 2.7-2.8 (m, 2H), 2.9-3.0 (m, 2H), 3.1-3.2 (m, 2H), 3.3-3.4 (m, 2H), 7.15-7.3 (m, 2H), 7.4-7.5 (m, 1H), 7.9-7.95 (m, 1H), 11.6-11.8 (m, 1H)) IR: 3400, 2900, 2500, 1680, 1600, 1440, 1360, 1300, 1220, 1150, 1110, 1060, 980, 910, 760, 735 cm⁻¹

### EXAMPLE 5

### 1′-phenylethyl-3,4-Dihydro-1-oxospiro[naphthalene-2(1H),4′-piperidine]

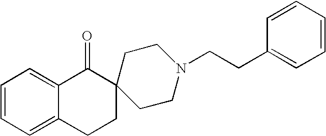

The method described for Example 2, using phenethyl bromide, results in the product in the hydrochloride form. Beige powder, M.p.=>275° C., Yd=55%.

TLC: (95/5 CH₂Cl₂/MeOH containing 10% NH₄OH): R_{f}=0.85. Analysis conforms to C₂₂H₂₆ClNO. N.M.R.: CDCl₃ ¹H ((ppm) HCl: 2.0-2.15 (m, 4H), 2.3-2.45 (m, 2H), 2.9-3.0 (m, 2H), 3.05-3.25 (m, 6H), 3.4-3.5 (m, 2H), 7.1-7.3 (m, 7H), 7.4-7.45 (m, 1H), 7.85-7.9 (m, 1H), 12.25-12.45 (m, 1H)) IR: 3400, 2900, 2500, 1670, 1600, 1450, 1360, 1290, 1220, 1110, 1010, 960, 820, 800, 740, 700 cm⁻¹

### EXAMPLE 6

### 1′-cyclopropylethyl-3,4-Dihydro-1-oxospiro[naphthalene-2(1H),4′-piperidine]

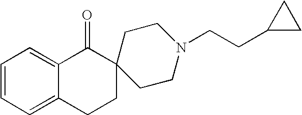

0.94 g (4.36 mmol) of 3,4-dihydro-1-oxospiro[naphthalene-2(1H),4′-piperidine] and then 10 ml of DMF are introduced into a three-necked flask. 1.3 g (8.7 mmol) of (bromoethyl)cyclopropane in 2 ml of DMF and then 0.73 g (8.7 mmol) of NaHCO₃ are added to the solution obtained. The suspension is brought to reflux and then maintained for 1.5 h. The solvents are removed at 50° C. under a vacuum of less than 1 mm Hg. The residue is taken up in 50 ml of water and extracted with three times 50 ml of ether. The ethereal phase is extracted with 100 ml of 1N HCl and then twice with 50 ml of water. The aqueous phase is basified while cold with concentrated NaOH and extracted 3 times with 50 ml of ether. The organic phase is washed with an NaCl solution and dried over Na₂SO₄. The solvent is removed. The oily residue is purified by fast chromatography by eluting with CH₂Cl₂ enriched with methanol. 0.6 g is obtained, the hydrochloride of which is prepared by addition of approximately 5N ethereal hydrochloric acid to a solution of the crude product in CH₂Cl₂. The mixture is concentrated to dryness and then the product is crystallized by addition of 30 ml of ether to a solution of the product in 5 ml of isopropanol. Crystallization is allowed to take place for 14 h at 20-25° C. and then the product is filtered off and washed with ether. After drying, 0.5 g of white powder is obtained, M.p.=244° C.,

TLC: (95/5 CH₂Cl₂/MeOH containing 10% NH₄OH): R_{f}=0.35. Analysis conforms to C₁₉H₂₆ClNO. N.M.R.: CDCl₃ ¹H ((ppm) HCl: 0.1-0.3 (m, 2H), 0.45-0.65 (m, 2H), 0.7-0.8 (m, 1H), 1.6-2.6 (m, 8H), 2.9-3.5 (m, 8H), 7.2-7.4 (m, 2H), 7.4-7.6 (t, 1H), 7.9-8.05 (d, 1H), 12.15 (1H)) IR: 2900, 2450, 1670, 1600, 1430, 1290, 1220, 950, 890, 740 cm⁻¹

### EXAMPLE 7

### 1′-cinnamyl-3,4-Dihydro-1-oxospiro[naphthalene-2(1H),4′-piperidine]

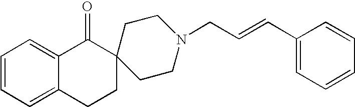

Prepared according to the method described in Example 2 with cinnamyl bromide, then purification by chromatography and crystallization of the hydrochloride.

White powder, M.p.=228° C. TLC: (95/5 CH₂Cl₂/MeOH containing 10% NH₄OH): R_{f}=0.55. Analysis conforms to C₂₃H₂₆ClNO. N.M.R.: CDCl₃ ¹H ((ppm) HCl: 2.0-2.25 (m, 4H), 2.3-2.5 (m, 2H), 2.9-3.85 (m, 8H), 6.4-6.6 (m, 1H), 6.6-6.8 (d, 1H), 7.1-7.6 (m, 8H), 7.9-8.0 (d, 1H), 12.1 (1H)) IR: 2900, 2400, 1670, 1590, 1420, 1290, 1220, 970, 730, 690 cm⁻¹

### EXAMPLE 8

### 1′-(3,3-diphenylpropyl)-3,4-Dihydro-1-oxospiro[naphthalene-2(11H),4′-piperidine]

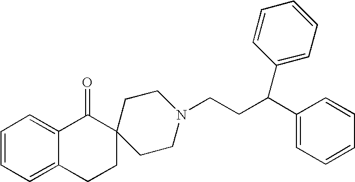

Prepared according to the method described in Example 6 with 3,3-diphenylpropyl bromide and preparation of the hydrochloride. A white powder is obtained, M.p.=257° C., TLC (95/5 CH₂Cl₂/MeOH): R_{f}=0.35. Analysis conforms to C₂₉H₃₂ClNO. N.M.R.: CDCl₃ ¹H ((ppm) HCl: 2.0-2.2 (m, 4H), 2.4-3.5 (m, 12H), 3.9-4.05 (m, 1H), 7.2-7.4 (m, 12H), 7.4-7.6 (m, 1H), 7.9-8.0 (m, 1H), 12.3 (1H)) IR: 2900, 2350, 1670, 1590, 1450, 1300, 1220, 910, 740, 700 cm⁻¹

### EXAMPLE 9

### 1′-(cyclopropylmethyl)-3,4-Dihydro-5,7-dimethyl-1-oxospiro[naphthalene-2(1H),4′-piperidine]

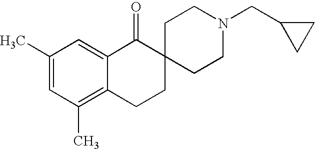

3,4-Dihydro-5,7-dimethyl-1-oxospiro[naphthalene-2(1H),4′-piperidine] is prepared according to the methods described for the synthesis of Example 1. The “N” alkylation is identical to that described in Example 6. The hydrochloride is obtained in the form of a white powder, M.p. >260° C.

TLC: (95/5 CH₂Cl₂/MeOH containing 10% NH₄OH): Rf=0.5. Analysis conforms to C₂₀H₂₈ClNO. N.M.R.: CDCl₃ ¹H ((ppm) HCl: 0.3-0.4 (m, 2H), 0.7-0.8 (m, 2H), 1.1-1.3 (m, 1H), 2.0-2.1 (m, 4H), 2.2 (s, 3H), 2.3 (s, 3H), 2.35-2.45 (m, 2H), 2.7-2.85 (m, 4H), 3.0-3.2 (m, 2H), 3.4-3.5 (m, 2H), 7.15 (s, 1H), 7.6 (s, 1H), 12.15 (1H)) IR: 3400, 2900, 2500, 1670, 1605, 1470, 1430, 1280, 1180, 1020, 970, 950, 880, 830 cm⁻¹

### EXAMPLE 10

### 1′-(cyclopropylmethyl)-3,4-Dihydro-6-methoxy-1-oxospiro[naphthalene-2(1H),4′-piperidine]

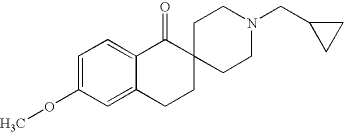

By the process of Example 9, the hydrochloride is obtained.

White powder, M.p. >255° C. TLC (90/10 CH₂Cl₂/MeOH containing 10% NH₄OH): Rf=0.35. Analysis conforms to C₁₉H₂₆ClNO₂. N.M.R.: CDCl₃ ¹H ((ppm) HCl: 0.4-0.5 (m, 2H), 0.75-0.85 (m, 2H), 1.25-1.4 (m, 1H), 2.05-2.2 (m, 4H), 2.35-2.5 (m, 2H), 2.8-2.95 (m, 2H), 2.95-3.05 (m, 2H), 3.2-3.4 (m, 2H), 3.45-3.6 (m, 2H), 3.85 (s, 3H), 6.7 (s, 1H), 6.85 (d, 1H), 7.9-8.0 (d, 1H), 12.15 (1H)) IR: 2900, 2420, 1660, 1590, 1430, 1250, 1220, 960, 830, 600 cm¹

### EXAMPLE 11

### 1′-(cyclopropylmethyl)-3,4-Dihydro-5-methoxy-1-oxospiro[naphthalene-2(1H),4′-piperidine]

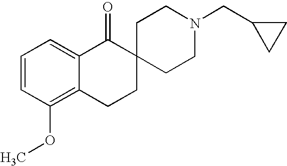

Same process as for Example 10; the hydrochloride is obtained.

White powder, M.p.=244° C. TLC (90/10 CH₂Cl₂/MeOH containing 10% NH₄OH): Rf=0.75. Analysis conforms to C₁₉H₂₆ClNO₂. N.M.R.: CDCl₃ ¹H ((ppm) HCl: 0.4-0.5 (m, 2H), 0.75-0.85 (m, 2H), 1.25-1.4 (m, 1H), 2.05-2.2 (m, 4H), 2.35-2.5 (m, 2H), 2.8-3.0 (m, 4H), 3.1-3.3 (m, 2H), 3.5-3.6 (m, 2H), 3.85 (s, 3H), 7.0-7.1 (m, 1H), 7.25-7.35 (m, 1H), 7.5-7.6 (m, 1H), 12.1-12.2 (1H)) IR: 2930, 2560, 2360, 1680, 1580, 1470, 1435, 1260, 1060, 970, 750 cm¹

### EXAMPLE 12

### 1′-(methylcyclo-propyl)-3,4-Dihydro-7-methoxy-1-oxospiro[naphthalene-2(1H),4′-piperidine]

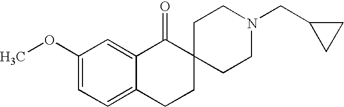

By using the same methods as for Example 10, the hydrochloride is obtained.

White powder, M.p.=235° C. TLC (90/10 CH₂Cl₂/MeOH containing 10% NH₄OH): R_{f}=0.70-0.75. Analysis conforms to C₁₉H₂₆ClNO₂. N.M.R.: CDCl₃ ¹H ((ppm) HCl: 0.4-0.5 (m, 2H), 0.75-0.85 (m, 2H), 1.25-1.4 (m, 1H), 2.05-2.2 (m, 4H), 2.35-2.5 (m, 2H), 2.8-3.0 (m, 4H), 3.2-3.3 (m, 2H), 3.5-3.6 (m, 2H), 3.80 (s, 3H), 7.0-7.1 (m, 1H), 7.15-7.25 (m, 1H), 7.4 (s, 1H), 12.1-12.2 (1H)) IR: 2930, 2510, 2445, 1670, 1610, 1495, 1415, 1250, 1025 cm⁻¹

### EXAMPLE 13

### 1′-(cyclopropylmethyl)-3,4-Dihydro-7-nitro-1-oxospiro[naphthalene-2(1H),4′-piperidine]

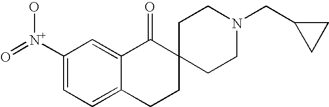

Stage 1: 2.4 g (8.35 mmol) of ethyl 3,4-dihydro-1-oxospiro[naphthalene-2(1H),4′-piperidine]-1′-carboxylate, prepared according to the method described in Stage 3 of Example 1, and 35 ml of concentrated sulphuric acid are introduced into a three-necked flask. 0.79 g (12.5 mmol) of fuming nitric acid is added to the solution cooled to 0° C. The mixture is stirred for 1 h at 0° C. and then for 2 h at 20-25° C. The solution is precipitated from 100 ml of water and ice and then extracted with three times 100 ml of CH₂Cl₂. The organic phase is washed successively with water and saturated NaCl solution. After drying over Na₂SO₄ and evaporating the solvent, 2.75 g of an oil are obtained, which oil is chromatographed by eluting with CH₂Cl₂ gradually enriched with acetone. 1.5 g of ethyl 3,4-dihydro-7-nitro-1-oxospiro[naphthalene-2(1H), 4′-piperidine]-1′-carboxylate are obtained in the form of an oily residue which crystallizes. Yd=54%, TLC (98/2 CH₂Cl₂/acetone): Rf=0.3.

N.M.R.: CDCl₃ ¹H ((ppm): 1.15 (t, 3H), 1.4 (m, 2H), 1.8-2.0 (m, 4H), 2.9-3.0 (m, 2H), 3.45-3.55 (m, 4H), 4.0-4.1 (q, 2H), 7.3 (d, 1H), 8.2 (d, 1H), 8.8 (s, 1H))

Stage 2: 1.5 g of the product of the preceding stage is hydrolysed by the process described in Stage 4, Example 1. After chromatography by eluting with CH₂Cl₂ gradually enriched with methanol containing 10% NH₄OH, 0.45 g is isolated. TLC (90/10 CH₂Cl₂/MeOH containing 10% NH₄OH): Rf=0.1.

Stage 3: 0.22 g (0.845 mmol) of 3,4-dihydro-7-nitro-1-oxospiro[naphthalene-2(1H),4′-piperidine] obtained in the preceding stage is suspended in 3 ml of acetonitrile. A solution of 0.343 g (2.54 mmol) of cyclopropylmethyl bromide in 0.5 ml of acetonitrile is added with stirring. The reaction mixture is brought to reflux and maintained for approximately 5 h.

The solvent is removed and the residue is taken up in 20 ml of CH₂Cl₂ and extracted with 20 ml of N/1 HCl. The acidic phase is basified while cold with a dilute sodium hydroxide solution to pH 12 and extracted with 3 times 20 ml of CH₂Cl₂. After washing, drying and removing the solvent, the residue is chromatographed by eluting with CH₂Cl₂ gradually enriched with methanol containing 10% NH₄OH. 0.130 g of 3,4-dihydro-1′-(cyclopropylmethyl)-7-nitro-1-oxospiro[naphthalene-2(1H),4′-piperidine] is obtained. TLC (90/10 CH₂Cl₂/MeOH containing 10% NH₄OH): Rf=0.7.

The hydrochloride is prepared as described above. White powder, M.p.=256° C. TLC (90/10 CH₂Cl₂/MeOH containing 10% NH₄OH): Rf=0.7. Analysis conforms to C₁₈H₂₃ClN₂O₃. N.M.R.: CDCl₃ ¹H ((ppm) HCl: 0.4-0.5 (m, 2H), 0.7-0.8 (m, 2H), 1.2-1.4 (m, 1H), 2-2.25 (m, 4H), 2.4-2.6 (m, 2H), 2.8-2.9 (m, 2H), 3.05-3.3 (m, 4H), 4.5-4.6 (m, 2H), 7.45 (d, 1H), 8.3 (d, 1H), 8.8 (s, 1H), 12.1 (1H)) IR: 2940, 2500, 2440, 1690, 1610, 1520, 1410, 1345, 1220, 1105, 960 cm⁻¹

### EXAMPLE 14

### 1′-(cyclopropylmethyl)-7-Amino-3,4-dihydro-1-oxospiro[naphthalene-2(1H),4′-piperidine]

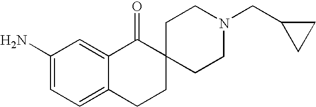

60 mg (0.19 mmol) of 3,4-dihydro-1′-(methylcyclo-propyl)-7-nitro-1-oxospiro[naphthalene-2(1H),4′-piperidine], obtained as described in Example 13, are dissolved in 1 ml of THF and then, with stirring, 0.21 g of tin chloride hydrate is introduced. The solution is brought to reflux for 1 h. The reaction liquors are charged to a saturated NaHCO₃ solution and extracted with 3 times CH₂Cl₂. The organic phase is washed and dried over Na₂SO₄. The solvent is evaporated and the residue obtained is chromatographed by eluting with CH₂Cl₂ gradually enriched with methanol containing 10% NH₄OH. 29 mg of product are obtained.

N.M.R.: CDCl₃ ¹H ((ppm): 0.1-0.2 (m, 2H), 0.4-0.55 (m, 2H), 0.85-1.0 (m, 1H), 1.6-1.7 (m, 2H), 1.9-2.1 (m, 4H), 2.3-2.4 (m, 2H), 2.5-2.75 (m, 4H), 2.8-2.9 (m, 2H), 3.6-3.8 (2H), 6.8 (d, 1H), 7.0 (d, 1H), 7.2 (s, 1H))

The hydrochloride is crystallized from ether. 26 mg of a yellow powder are obtained.

M.p.=200° C., decomposition. TLC (95/5 CH₂Cl₂/MeOH containing 10% NH₄OH): Rf=0.45.

### EXAMPLE 15

### 1′-(cyclopropylmethyl)-7-Chloro-3,4-dihydro-1-oxospiro[naphthalene-2(1H),4′-piperidine]

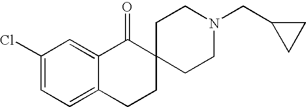

Stage 1: Piperidine-1,4-dicarboxylic acid, 4-ethyl 1-t-butyl Diester.

60 g (0.381 mol) of ethyl isonipecotate and 400 ml of THF are placed in a three-necked flask which is protected from moisture and which is under an inert atmosphere, and 18.3 g (0.458 mol) of sodium hydroxide pellets are added. A solution of 100 g (0.458 mol) of di-t-butyl dicarbonate in 170 ml of THF is added over 1 h with stirring to the suspension. The temperature reaches 45° C. The reaction mixture is left stirring for 14 h at 20-25° C. and is then poured onto 2 l of water and ice and extracted with 3 times 500 ml of ether. The organic phase is washed with 3 times 250 ml of a saturated NaCl solution, dried over Na₂SO₄ and concentrated. The residue is chromatographed by eluting with CH₂Cl₂ gradually enriched with acetone and then distilled under a vacuum of 0.09 mm Hg and at a vapour temperature of 95-102° C. 82 g are obtained (Yd=83.6%). TLC (95/5 CH₂Cl₂/acetone): Rf=0.60.

N.M.R.: CDCl₃ ¹H ((ppm): 1.2-1.3 (t, 3H), 1.4 (s, 9H), 1.5-1.6 (m, 2H), 1.8-1.9 (m, 2H), 2.35-2.45 (m, 1H), 2.7-2.85 (m, 2H), 3.9-4.0 (m, 2H), 4.05-4.15 (q, 2H))

Stage 2: 4-(4-Chlorophenethyl)piperidine-1,4-dicarboxylic acid, 4-ethyl 1-t-butyl Diester

6.16 g (60.9 mmol) of diisopropylamine and 174 ml of THF, dried over molecular sieve, are introduced, by transfer under nitrogen, into a three-necked flask which is protected from moisture and which is under an inert atmosphere. The solution is cooled to −10° C. and 24.3 ml of 2.5N n-butyllithium in hexane (60.9 mmol) are run in. The mixture is stirred for 15 min at −10° C. and cooled to −70° C., and a solution of 10.4 g (40.6 mmol) of the product from the preceding Stage 1 in 86 ml of THF is run in over approximately 20 min. The mixture is stirred for 10 min at −70° C. and then 10.9 g (60.9 mmol) of HMPT are added. The mixture is kept stirring at −70° C. for 1.5 h and a solution of 4-chlorophenethyl bromide (10.7 g, 48.7 mmol) in 86 ml of THF is run in over 20 min at −70° C. The mixture is stirred at 20-25° C. for 14 h and then poured over 350 ml of water and extracted 3 times with ether. The organic phase is washed with an N/1 HCl solution and then with a saturated NaCl solution. After drying and concentrating, 17 g of an orange oil are obtained, which oil is chromatographed by eluting with CH₂Cl₂ gradually enriched with hexane, and then with acetone. 11.8 g are obtained (Yd=80%). TLC (95/5 CH₂Cl₂/acetone): Rf=0.70.

NMR: CDCl₃ ¹H ((ppm): 1.2-1.3 (t, 3H), 1.4 (s, 9H), 1.3-1.4 (m, 2H), 1.7-1.8 (m, 2H), 2.0-2.1 (m, 2H), 2.3-2.4 (m, 2H), 2.7-2.9 (m, 2H), 3.7-3.9 (m, 2H), 4.05-4.15 (q, 2H), 6.9-7.0 (m, 2H), 7.1-7.2 (m, 2H))

Stage 3: Ethyl 4-(4-chlorophenethyl)piperidine-4-carboxylate

10.8 g of the product from the preceding Stage 2 and 50 ml of CH₂Cl₂ are introduced into a three-necked flask which is protected from moisture. The solution is stirred and 25 ml of trifluoroacetic acid are added at 20-25° C. The mixture is kept stirring for 30 min and then concentrated to dryness and the residue is taken up in ether. The organic phase is washed with a 10% sodium hydroxide solution and then with a saturated NaCl solution. After drying and concentrating, 9 g of an oil are obtained, which oil crystallizes.

TLC (90/10 CH₂Cl₂/MeOH containing 10% NH₄OH): Rf=0.45.

NMR: CDCl₃ ¹H ((ppm): 1.1-1.25 (t, 3H), 1.3-1.4 (m, 2H), 1.7-1.8 (m, 2H), 2.1-2.2 (m, 2H), 2.35-2.45 (m, 2H), 2.6-2.7 (m, 2H), 2.9-3.0 (m, 2H), 3.3 (1H), 4.1-4.2 (q, 2H), 6.9-7.0 (dd, 2H), 7.1-7.2 (dd, 2H))

Stage 4: Ethyl 1-(cyclopropylmethyl)-4-(4-chlorophenethyl)piperidine-4-Carboxylate

3.4 g (11.5 mmol) of the product from the preceding Stage 3, 85 ml of THF, dried over molecular sieve, and then, with stirring, 14.9 ml of triethylamine and 2.4 ml (20.9 mmol) of 85% (bromomethyl)cyclopropane are successively introduced into a round-bottomed flask which is protected from moisture and which is under nitrogen. The mixture is brought to reflux for 14 h and then concentrated to dryness, and the residue is taken up in water and extracted twice with ether. The organic phase, washed with a saturated NaCl solution and dried, is concentrated. 3 g of crude product are obtained, which product is chromatographed (eluent: CH₂Cl₂ gradually enriched with methanol containing 10% NH₄OH). 2.6 g of oily product are obtained. Yd=65%.

TLC (95/5 CH₂Cl₂/MeOH containing 10% NH₄OH): Rf=0.50. N.M.R.: CDCl₃ ¹H ((ppm): 0.0-0.1 (m, 2H), 0.35-0.45 (m, 2H), 0.7-0.8 (m, 1H), 1.1-1.2 (t, 3H), 1.4-1.5 (m, 2H), 1.7-1.8 (m, 2H), 1.9-2.1 (m, 2H), 2.1-2.2 (m, 2H), 2.3-2.4 (m, 2H), 2.7-2.85 (m, 2H), 4.1-4.2 (q, 2H), 6.9-7.0 (dd, 2H), 7.1-7.2 (dd, 2H))

Stage 5: 1-(Cyclopropylmethyl)-4-(4-chlorophenethyl)-piperidine-4-carboxylic Acid

2.2 g (6.28 mmol) of the preceding ester and 6.6 ml of anhydrous dimethyl sulphoxide are introduced into a round-bottomed flask which is protected from moisture and which is under nitrogen. A solution of potassium t-butoxide (4.4 g, 39 mmol) in 30 ml of dimethyl sulphoxide is added with stirring. The mixture is left stirring for 2 h at 20-25° C. The reaction mixture is charged to 200 ml of water and then washed with ether. The aqueous phase is acidified to pH 5-7 with 10% HCl. The precipitate is filtered off and washed with water. The acid obtained is crystallized from a CH₂Cl₂/methanol mixture. M.p.=250° C.

NMR: CDCl₃ ¹H ((ppm): 0.2-0.3 (m, 2H), 0.5-0.6 (m, 2H), 1.07-1.1 (m, 1H), 1.6-1.9 (m, 4H), 2.25-2.35 (m, 2H), 2.5-2.6 (m, 2H), 2.65-2.75 (m, 2H), 2.8-2.9 (m, 2H), 3.3-3.4 (m, 2H), 6.9-7.0 (dd, 2H), 7.1-7.2 (dd, 2H)) IR: 3370, 1490, 1445, 1380, 1240, 1170, 1095, 965, 805 cm⁻¹.

Stage 6: 7-Chloro-3,4-dihydro-1′-(cyclopropylmethyl)-1-oxospiro[naphthalene-2(1H),4′-piperidine].

0.3 g (0.9 mmol) of the acid obtained previously and 6 ml of benzene are introduced into a round-bottomed flask which is protected from moisture and which is under nitrogen. 0.24 g of PCl₅ and 6 ml of CH₂Cl₂ are added, followed by a further 0.24 g of PCl₅. The mixture is stirred for 2 h at 20-25° C. The mixture is cooled to 0° C., 0.44 ml of tin tetrachloride is introduced (copious precipitation), 12 ml of CH₂Cl₂ are added and the mixture is maintained at 0° C. for 1 h and then at 20-25° C. for 14 h. The solvents are removed and the residue is taken up in water. The aqueous phase is washed with ether and then basified to pH 12 with NaOH and extracted with ether. The organic phase is washed, dried and concentrated. The crude product is chromatographed by eluting with CH₂Cl₂ gradually enriched with methanol containing 10% NH₄OH. 22 mg of product are obtained, which product is treated in solution in CH₂Cl₂ with 5N ethereal hydrochloric acid. After crystallization from ethyl acetate, the product is filtered off and dried at 50° C. under vacuum.

White powder. TLC (90/10 CH₂Cl₂/MeOH containing 10% NH₄OH): Rf=0.55. M.p.=263° C. N.M.R.: CDCl₃ ¹H ((ppm) HCl: 0.4-0.5 (m, 2H), 0.7-0.8 (m, 2H), 1.2-1.3 (m, 1H), 2.0-2.15 (m, 4H), 2.4-2.55 (m, 2H), 2.8-2.9 (m, 2H), 2.95-3.05 (m, 2H), 3.1-3.3 (m, 2H), 3.5-3.6 (m, 2H), 7.2-7.3 (m, 1H), 7.4-7.5 (m, 1H), 7.9 (s, 1H), 12.2 (1H)) IR: 2930, 2440, 1720, 1490, 1230, 1185, 1095, 1025, 810 cm⁻¹.

### EXAMPLES 16-34

Following the general procedures described above, the following additional compounds listed in table 1 were prepared.

TABLE 1

| Ex- | | | | | | |
|---|---|---|---|---|---|---|
| ample | R¹ | R² | R³ | R⁴ | A | R⁵ |
| 16 | ═O | | H | H | CH₂ | CH₃ |
| 17 | ═O | | H | H | CH₂ | CH₂CH═CH₂ |
| 18 | ═O | | H | H | CH₂ | 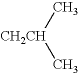 |
| 19 | ═O | | H | H | CH₂ | 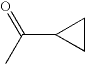 |
| 20 | ═O | | H | H | CH₂ | 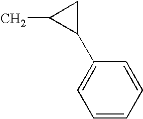 |
| 21 | ═O | | H | H | CH₂ | 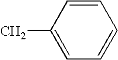 |
| 22 | ═O | | H | H | CH₂ | 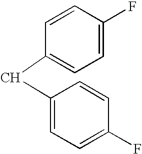 |
| 23 | H | OH | H | H | CH₂ | 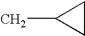 |
| 24 | H | H | H | H | CH₂ | 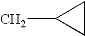 |
| 25 | ═O | | H | H | Bond | 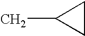 |
| 26 | ═O | | H | H | CH_{2 CH}₂ | 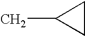 |
| 27 | ═O | | H | H | 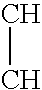 | 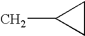 |
| 28 | ═O | | 6-Cl | H | CH₂ | 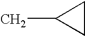 |
| 29 | ═O | | 6-F | H | CH₂ | 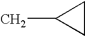 |
| 30 | ═O | | 6:OCH₃ | 7:OCH₃ | CH₂ | 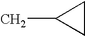 |
| 31 | ═O | | H | H | CH₂ | 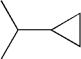 |
| 32 | ═O | | H | H | CH₂ | 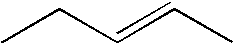 |
| 33 | ═O | | H | H | CH₂ | 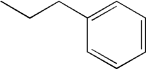 |
| 34 | ═O | | 6:OCH₃ | H | CH₂ | 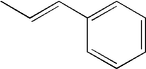 |

MP and NMR data for the compounds of examples 16 to 34 are provided below:

### EXAMPLE 16

MP=240-243° C. R.M.N. CDCl₃ ¹H δ (ppm) Base:1.5-1.6 (m,2H); 1.95-2.05 (m,4H); 2.25 (s,3H); 2.3-2.4 (m,2H); 2.45-2.55 (m,2H); 2.9-2.95 (m,2H); 7.1-7.15 (m,1H); 7.2-7.25(m,1H); 7.35-7.4 (m,1H); 7.9-7.95 (m,1H)

### EXAMPLE 17

MP=242-244° C.

R.M.N. CDCl₃ ¹H δ (ppm) HCl:2.05-2.15 (m,4H); 2.3-2.45 (m,2H); 3.0-3.1 (m,2H); 3.1-3.25 (m,2H); 3.35-3.5 (m,2H); 3.55-3.6 (m,2H); 5.4-5.55 (m,2H); 6.1-6.25(m,1H); 7.2-7.35 (m,2H); 7.45-7.5 (m,1H); 7.9-7.95 (m,1H); 12.3-12.45 (m,1H)

### EXAMPLE 18

MP=244-245° C. R.M.N. CDCl₃ ¹H δ (ppm) HCl: 1.15 (d,6H); 2.0-2.1 (m,4H); 2.15-2.3 (m,1H); 2.55-2.65 (m,2H); 2.75-2.8 (m,2H); 2.95-3.0 (m,2H); 3.1-3.25 (m,2H); 3.35-3.45 (m,2H);7.2-7.35 (m,2H); 7.45-7.5 (m,1H); 7.9-7.95 (m,1H); 11.7-11.8 (m,1H)

### EXAMPLE 19

MP=95-97° C. R.M.N. CDCl₃ ¹H δ (ppm) base: 0.6-0.7 (m,2H); 0.8-0.95 (m,2H); 1.4-1.5 (m,2H); 1.6-1.7 (m,1H); 1.8-2.1 (m,4H); 2.9-3.5 (m,2H); 3.4-3.8 (m,4H); 7.1-7.3 (m,2H); 7.35-7.45 (m,1H); 7.9-8,0 (m,1H);

### EXAMPLE 20

MP=220-221° C. R.M.N. CDCl₃ ¹H ε (ppm) HCl: 1.05-1.3 (m,2H); 1.5-1.9 (m,1H); 2.0-2.2 (m,5H); 2.3-2.55 (m,2H); 2.9-3.4 (m,6H); 3.4-3.6 (m,2H); 7.0-7.6 (m,8H); 7.9-8,1 (m,1H); 12.2-12.4 (m,1H)

### EXAMPLE 21

MP=261-262° C. R.M.N. CDCl₃ ¹H δ (ppm) HCl: 2.0-2.1 (m,4H); 2.4-2.5 (m,2H); 2.95-3.05 (m,2H); 3.1-3.25 (m,2H); 3.3-3.4 (m,2H); 4,1-4,15 (m,2H); 7.2-7.35 (m,2H); 7.4-7.5 (m,4H); 7.65-7.7 (m,2H); 7.9-7.95 (m, 1H); 12.25-12.5 (m,1H)

### EXAMPLE 22

MP=>250° C. R.M.N. CDCl₃ ¹H δ (ppm) base: 1.4-1.55 (m,2H); 1.9-2.05(m,4H); 2.2-2.35 (m,2H); 2.4-2.55 (m,2H); 2.8-2.95 (m,2H); 4,2 (s,1H); 6.8-7.0 (m,4H); 7.15 (d,1H); 7.2-7.35 (m,5H); 7.4 (t,1H); 7.9 (d,1H)

### EXAMPLE 23

MP=91-93° C. R.M.N. CDCl₃ ¹H δ (ppm) base: −0.05-0.05 (m,2H); 0.35-0.45(m,2H); 0.7-0.85 (m,1H); 1.25-1.85 (m,7H); 2.2 (d,2H); 2.2-2.4 (m,2H); 2.5-2.6 (m,1H); 2.6-2.7 (m,3H); 4,2 (s,1H); 7.0-7.3 (m,4H)

### EXAMPLE 24

MP=256-258° C. R.M.N. CDCl₃ ¹H ε (ppm) base: 0.0-0.1 (m,2H); 0.4-0.5(m,2H); 0.75-0.85 (m,1H); 1.45-1.55 (m,4H); 1.6-1.65 (m,2H); 2.2 (d,2H); 2.35-2.6 (m,6H); 2.7-2.8 (m,2H); 6.9-7.05 (m,4H)

### EXAMPLE 25

MP=241° C. R.M.N. CDCl₃ ¹H δ (ppm) HCl: 0.0-0.15 (m,2H); 0.4-0.55 (m,2H); 0.75-0.9 (m,1H); 1.35 (d,2H); 1.9-2.15 (m,4H); 2.25 (d,2H); 2.9 (s,2H); 3.0 (d,2H); 7.25 (t,1H); 7.35 (d 1H); 7.5 (t,1H); 7.65 (d,1H); 12.1-12.2 (1H)

### EXAMPLE 26

MP=242-243° C. R.M.N. CDCl₃ ¹H δ (ppm) HCl: 0.4-0.5 (m,2H); 0.7-0.8 (m,2H); 1.2-1.35 (m,1H); 1.85-2.0 (m,4H); 2.2-2.3 (m,2H); 2.3-2.4 (m,2H); 2.7-2.9 (m,6H); 3.4-3.5 (m,2H); 7.15-7.4 (m,4H);12.1-12.3 (1H)

### EXAMPLE 27

MP=234° C. R.M.N. CDCl₃ ¹H δ (ppm) HCl: 0.45 (m,2H); 0.75 (m,2H); 1.35 (m,1H); 1.4(dd,3H); 1.85 (m,2H); 2.1 (m,3H); 2.3 (m,1H); 2.75 (m,2H); 2.9 (m, 2H) 3.25 (m,1H); 3.45-3.8 (m,3H); 7.3-7.45 (m,2H);7.6 (m,1H); 8,0 (dd,1H); 12.1 (1H)

### EXAMPLE 28

MP=>250° C. R.M.N. CDCl₃ ¹H δ (ppm) HCl: 0.4-0.5 (m,2H); 0.75-0.85 (m,2H); 1.3-1.4 (m,1H); 2.1-2.2 (m,4H); 2.4-2.55 (m,2H); 2.9-2.95 (m,2H); 3.0-3.05 (m, 2H); 3.2-3.3 (m,2H); 3.5-3.6 (m,2H); 7.25-7.3 (m,2H);7.9,7.95 (m,1H); 12.2 (1H)

### EXAMPLE 29

MP=227° C. R.M.N. CDCl₃ ¹H δ (ppm) HCl: 0.4-0.5 (m,2H); 0.75-0.85 (m,2H); 1.3-1.4 (m,1H); 2.0-2.2 (m,4H); 2.4-2.6 (m,2H); 2.85-2.95 (m,2H); 3.0-3.1 (m, 2H); 3.2-3.3 (m,2H); 3.5-3.6 (m,2H); 6.9-7.0 (m,1H); 7.0-7.1 (m,1H); 7.95-8,05 (m,1H); 12.1 (1H)

### EXAMPLE 30

MP=229° C. R.M.N. CDCl₃ ¹H δ (ppm) HCl: 0.45 (m,2H); 0.75 (m,2H); 1.35 (m,1H); 2.15 (m,4H); 2.45 (td,2H); 2.9 (m,2H); 2.95 (m, 2H); 3.3 (m,2H); 3.55 (m,2H); 4,85 (s,3H); 4,95 (s,3H); 6.65 (s,1H); 7.45 (s,1H); 12.05 (1H)

### EXAMPLE 31

MP=189-192° C. R.M.N. CDCl₃ ¹H δ (ppm) HCl: 0.2-0.3 (m,1H); 0.6-0.7 (m, H1); 0.7-0.8 (m,1H); 0.8-0.9 (m,1H); 1.1-1.2 (m,1H); 1.55 (d,3H); 1.6-1.7 (m,1H); 2.1-2.2 (m,4H); 2.5-2.7 (m,2H); 3.0-3.1 (m,2H); 3.3-3.6 (m,4H); 7.2-7.25 (m,1H); 7.3-7.35 (m,1H); 7.45-7.5 (m1H); 8,0-8,05 (m,1H); 11.85 (1H)

### EXAMPLE 32

MP=>235° C. R.M.N. CDCl₃ ¹H δ (ppm) HCl: 1.7 (d,3H); 2.1-2.2 (m,4H); 2.4-2.5 (m,2H); 2.6-2.7 (m,2H); 2.9-3.1 (m,4H); 3.2-3.3 (m,2H); 3.4-3.5 (m,2H); 5.3-5.4 (m,1H);5.6-5.7 (m,1H); 7.2-7.3 (m,1H); 7.3-7.35 (m,1H); 7.5-7.6 (m1H); 7.95-8,0 (m,1H); 12.2 (1H)

### EXAMPLE 33

MP=209° C. R.M.N. CDCl₃ ¹H δ (ppm) HCl: 2.1 (m,4H); 2.25 (m,2H); 2.45 (m,2H); 2.7 (m,2H); 2.9 (m, 2H); 3.0 (m,2H); 3.15 (m,2H); 3.4 (m,2H); 7.1-7.35 (m,7H); 7.5 (m,1H); 7.95 (m,1H); 11.95 (1H)

### EXAMPLE 34

MP=228° C. R.M.N. CDCl₃ ¹H δ (ppm) HCl: 2.0-2.2 (m,4H); 2.4 (td,2H); 3.0 (m,2H); 3.3 (qd, 2H); 3.45 (m,2H); 3.75 (t,2H); 3.85 (s,3H); 6.55 (qt,1H); 6.7 (m,2H); 6.85 (dd,1H); 7.35 (m,3H); 7.45 (d,2H); 7.95 (d,1H); 11.95 (1H).

### EXAMPLES 35-69

Following procedures known in the art, some of the tetralones described above were alkylated to yield the following additional compounds listed in table 2.

TABLE 2

| Ex. | R¹-R² | R₃ | R₄ | R₅ | A | Rf |
|---|---|---|---|---|---|---|
| 35 | ═O | 5:OCH₃ | H | (CH₂)₂-c-C₃H₅ | CH₂ | 0.7 |
| 36 | ═O | 5:OCH₃ | H | CH₂-c-C₄H₇ | CH₂ | 0.5 |
| 37 | ═O | 5:OCH₃ | H | CH₂-c-C₆H₁₁ | CH₂ | 0.8 |
| 38 | ═O | 5:OCH₃ | H | CH₂—CH═CH—C₆H₆ | CH₂ | 0.7 |
| 39 | ═O | 5:OCH₃ | H | CH₂—CH₂—C₆H₆ | CH₂ | 0.7 |
| 40 | ═O | 5:OCH₃ | H | CH₂—CH₂—CH₂—C₆H₆ | CH₂ | 0.55 |
| 41 | ═O | 5:OCH₃ | H | (CH₂)₂—CH—(C₆H₆)₂ | CH₂ | 0.90 |
| 42 | ═O | 6:OCH₃ | H | (CH₂)₂-c-C₃H₅ | CH₂ | 0.4 |
| 43 | ═O | 6:OCH₃ | H | CH₂-c-C₄H₇ | CH₂ | 0.3 |
| 44 | ═O | 6:OCH₃ | H | CH₂-c-C₆H₁₁ | CH₂ | 0.55 |
| 45 | ═O | 6:OCH₃ | H | CH₂—CH═CH—C₆H₆ | CH₂ | 0.5 |
| 46 | ═O | 6:OCH₃ | H | CH₂—CH₂—C₆H₆ | CH₂ | 0.5 |
| 47 | ═O | 6:OCH₃ | H | CH₂—CH₂—CH₂—C₆H₆ | CH₂ | 0.4 |
| 48 | ═O | 6:OCH₃ | H | (CH₂)₂—CH—(C₆H₆)₂ | CH₂ | 0.7 |
| 49 | ═O | 7:OCH₃ | H | (CH₂)₂-c-C₃H₅ | CH₂ | 0.5 |
| 50 | ═O | 7:OCH₃ | H | CH₂-c-C₄H₇ | CH₂ | 0.2 |
| 51 | ═O | 7:OCH₃ | H | CH₂-c-C₆H₁₁ | CH₂ | 0.4 |
| 52 | ═O | 7:OCH₃ | H | CH₂—CH═CH—C₆H₆ | CH₂ | 0.4 |
| 53 | ═O | 7:OCH₃ | H | CH₂—CH₂—C₆H₆ | CH₂ | 0.4 |
| 54 | ═O | 7:OCH₃ | H | CH₂—CH₂—CH₂—C₆H₆ | CH₂ | 0.3 |
| 55 | ═O | 7:OCH₃ | H | (CH₂)₂—CH—(C₆H₆)₂ | CH₂ | 0.65 |
| 56 | ═O | H | H | (CH₂)₂-c-C₃H₅ | CHCH₃ | 0.4 |
| 57 | ═O | H | H | CH₂-c-C₄H₇ | CHCH₃ | 0.2 |
| 58 | ═O | H | H | CH₂-c-C₆H₁₁ | CHCH₃ | 0.5 |
| 59 | ═O | H | H | CH₂—CH═CH—C₆H₆ | CHCH₃ | 0.45 |
| 60 | ═O | H | H | CH₂—CH₂—C₆H₆ | CHCH₃ | 0.5 |
| 61 | ═O | H | H | CH₂—CH₂—CH₂—C₆H₆ | CHCH₃ | 0.4 |
| 62 | ═O | H | H | (CH₂)₂—CH—(C₆H₆)₂ | CHCH₃ | 0.7 |
| 63 | ═O | 6:Cl | H | (CH₂)₂-c-C₃H₅ | CH₂ | 0.5 |
| 64 | ═O | 6:Cl | H | CH₂-c-C₄H₇ | CH₂ | 0.3 |
| 65 | ═O | 6:Cl | H | CH₂-c-C₆H₁₁ | CH₂ | 0.65 |
| 66 | ═O | 6:Cl | H | CH₂—CH═CH—C₆H₆ | CH₂ | 0.55 |
| 67 | ═O | 6:Cl | H | CH₂—CH₂—C₆H₆ | CH₂ | 0.65 |
| 68 | ═O | 6:Cl | H | CH₂—CH₂—CH₂—C₆H₆ | CH₂ | 0.5 |
| 69 | ═O | 6:Cl | H | (CH₂)₂—CH—(C₆H₆)₂ | CH₂ | 0.85 |

### EXAMPLE 70

### 3.4-Dihydro-6-methoxy-1-oxospiro[naphthalene-2(1H),4′-piperidine]

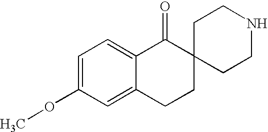

The synthesis is similar to the one described in example 1 except in stage 3 wherein 6-methoxytetralone is used instead of 1-tetralone. N.M.R.: CDCl₃ ¹H ((ppm) Base: 1.9-2.3 (m, 6H); 2.9-3.1 (m, 2H); 3.3-3.6 (m, 4H); 3.85 (s, 3H); 6.65 (s, 1H); 6.8-6.9 (m, 1H); 7.9-8.0 (dd, 1H); 9.5 (bs, 2H)). M.P.=236-237° C., TLC (90/10 CH₂Cl₂/MeOH containing 10% NH₄OH): R_{f}=0.25. IR: 2724, 1657. 1595. 1446. 1258, 1223. 1086. 978, 906. 839 cm⁻¹

As noted above, the invention compounds of Formula I are useful for treating chronic pain and other CNS disorders such as seizures, e.g. epilepsy. The compounds have been evaluated in standard assays to measure their ability to block isolated mammalian Na neuronal channels, as well as their ability to antagonize prostaglandin E₂ (PGE₂) production. Both assays are routinely utilized to indicate clinical utility of compounds for treating chronic pain and other CNS disorders (see Tonelian et al., Anesthesiology, 24: 949-951. 1991).

### EXAMPLE 71

### Sodium Channel [³H]Batrachotoxin (BTX) Binding Assay

Cerebral cortices from male Sprague-Dawley rats were homogenized in a glass-Teflon homogenizer in 10 volumes of ice-cold 0.32 M sucrose, 5 mM K₂HPO₄ (pH 7.4 at 4° C.). The homogenate was centrifuged at 1000 g. for 10 min, the pellet was resuspended in the same volume of sucrose and recentrifuged. The pellet was discarded and the two supernatants resulting from these two centrifugations were pooled and centrifuged at 20000 g. for 10 min. The resulting pellet was resuspended in a Na-free assay buffer containing 50 mM HEPES, 5.4 mM KCl, 0.8 mM MgSO₄, 5.5 mM glucose and 130 mM choline chloride (pH 7.4 at 25° C.). Binding assay were initiated by the addition of 150-200 μg synaptosomal protein to an assay buffer containing 25 μg scorpion venom (Leirus quinquestriatus), 0.1% BSA and 10 nM [³H] batrachotoxin (40 Ci/mmol, NEN) in the presence or absence of different concentrations of unabelled drugs (250 μl final volume). Non-specific binding was determined in the presence of 0.3 mM veratridine. Reactions were incubated for 90 min at 25° C. and bound ligand was separated from free by vacuum filtration through Whatman GF/B filters; the filters were washed with 2×5 ml buffer (5 mM HEPES, 1.8 mM CaCl₂, 0.8 mM MgSO₄, 130 mM choline chloride, 0.01% BSA; pH 7.4 at 25° C.) and bound ligand was estimated by liquid scintillation spectrometry.

### EXAMPLE 72

### ²²Na⁺ influx into SK-N-SH Neuroblastoma Cells

Characterization of Na⁺ channels activity is performed using human SK-N-SH cells in 96-well culture plates. The effect of tested compounds on Na⁺ influx through the Na⁺ channels is evaluated under stimulation by veratridine. SK-N-SH cells are preincubated for 15 min at 37° C. in the presence of test compounds in a 25 mM hepes/Tris pH 7.5 buffer containing 5.4 mM KCl, 0.8 mM MgSO₄, 1.8 mM CaCl₂, 5 mM glucose, 0.1% BSA, 140 mM choline chloride. The influx of Na⁺ is induced by the incubation for 10 min at 37° C. of SK-N-SH cells in the presence of test compound and veratridine in the incubation buffer supplemented with 1 μM ouabaine, 10 mM NaCl, 130 mM choline chloride and ²²Na⁺ (Jacques, Y, Fosset, M. and Lazdunski, M., (1978), Molecular properties of the action potential Na⁺ ionophore in neuroblastoma cells. J. Biol. Chem., 253. 7383-7392).

Following this ²²Na⁺ uptake, cells are washed with 0.1 mM MgCl₂. The radioactivity is then measured with a microplate reader (Topcount, Packard) after the addition of a scintillation liquid (Microscint 40, Packard).

The reference compound is tetrodotoxin tested at 7 concentrations ranging from 10⁻¹⁰ M to 10⁻⁷ M in order to determine an IC₅₀ value.

### EXAMPLE 73

### Analgesic Activity on Chronic Hyperalgesia Induced by PGE₂ in Rats

The test consists in determining the analgesic effect of the test compound in rats by the Randall and Selitto test, in which chronic hyperalgesia has been triggered by intraplantar injection of PGE₂ over 4 days into a leg, according to a protocol adapted from Nakamura-Craig et al (Pain, 63: 33-37. 1995).

The study is carried out on batches of 120-140 g Sprague-Dawley rats to which 100 ng of PGE₂ is administered in a volume of 100 μl by the intraplantar route, for 4 consecutive days twice a day; this causes chronic hyperalgesia in the leg from the 5th day, for at least one week. On the day of the test, in the morning, the threshold of reaction to pain is checked by the Randall and Selitto test, and animals whose threshold is ψ100 arbitrarily defined units are selected. In the afternoon, the measurement is repeated after prior administration by the s.c. route of a solution of the test compound; this administration is carried out 30 min before measuring the pain threshold. For each batch, the analgesic activity (%) is calculated from the means of the thresholds measured before and after treatment, as compared with that of the control animals, who received only the vehicle.

The following Table 3 lists the Na channel binding and the analgesic activities of representative compounds of the invention when measured in the foregoing assays.

TABLE 3
Pharmacological activities

| | [³H] BTX assay: | | PGE₂ assay: |
|---|---|---|---|
| Compound of | Na⁺ channel | ²²Na influx | Analgesic activity at |
| Example No | binding Ki (nM) | IC50 (μM) | 10 mg/kg S.C. (%) |
| 2 | 876 | 10.7 | 49 |
| 6 | 1435 | 3.4 | 67 |
| 7 | 366 | 0.94 | 57 |
| 8 | 397 | 0.97 | 47 |
| 10 | 3890 | 30 | 100 |
| 33 | 291 | 1.2 | 81 |
| 36 | 475 | 3.4 | 51 |
| 42 | ND | 4.3 | 46 |
| 43 | ND | 6 | 39 |
| 47 | ND | 0.6 | 66 |
| 49 | 2183 | 7.6 | 38 |
| 70 | ND | ND | 46 |

The foregoing biological data establish that the compounds of Formula I are particularly useful for treating CNS disorders in mammals, especially neuropathic pain, trigeminal neuralgia, diabetic neuropathy, sciatic neuropathy and seizures. The compounds are particularly well suited to the treatment of diabetic neuropathy, which is the most common complication accompanying diabetes mellitus. The compounds also are useful for prophylaxis and treatment of migraine.

The invention compounds can be administered to humans who are in need of treatment for a chronic pain condition or seizure disorder by both the oral and parenteral routes, for instance as tablets or capsules, or as subcutaneous or intravenous injections. The compounds will be administered in an amount which is effective to control and treat the seizure disorder or relieve the neuropathic pain sensation. Such effective amounts will generally be from about 0.1 to about 2000 mg/kg of mammalian body weight. Commonly prescribed doses will be from about 5 mg/kg to about 500 mg/kg. Such dosage amounts can be administered to adult humans from 1 to 4 times a day for the relief of neuropathic pain and seizure disorders. The precise dose to be employed will depend upon the specific compound of Formula I utilized, the particular condition of the subject being treated, and generally will be dictated by the attending physician or other medical practitioner.

The compounds can be formulated by normal methods for convenient oral or parenteral dosing. Typical oral forms are tablets, capsules, troches, elixirs, syrups, suspensions, and controlled sustained release forms, for example through osmotic pumps. The compounds can likewise be formulated for administration intraperitoneally, subcutaneously, intramuscularly, transdermally, sublingually or intravenously. The compounds are formulated by using conventional diluents, excipients, carriers and binders routinely used in the phamaceutical art. For example, the compounds can be admixed with carriers, diluents and excipients such as starch, cellulose, PVP, methylcellulose, sugar, wax, talc, and with stabilizers and binders such as Mg stearate, MgO, CaCO₃, methyl-p-hydroxybenzoate (methylparaben), and n-propyl-p-hydrobenzoate (propylparaben).

The following additional examples illustrate typical pharmaceutical formulations which are provided by this invention.

### EXAMPLE 74

Tablet Preparation

| | |
|---|---|
| Compound of Example 10 | 25.0 mg |
| Microcrystalline cellulose | 50.0 mg |
| Modified food corn starch | 50.0 mg |
| Magnesium stearate | 1.0 mg |

The above ingredients are blended to uniformity and compressed into a tablet. Such tablets are administered at the rate of 1 to 4 times a day to a human suffering from chronic pain.

### EXAMPLE 75

Intravenous Preparation

| | |
|---|---|
| Compound of example 2 | 400 mg |
| Acetate buffer | 20 ml |
| dil aqueous HCl or NaOH | to pH6.5 |
| Sterile isotonic saline | qs 1000 ml |

The invention compound is dissolved in the acetate buffer and the pH is adjusted to 6.5. Isotonic saline is added to a volume of 1000 ml. The solution is filled into a sterile flexible plastic container equipped with a drip tube. The solution is administered IV to a patient suffering from diabetic neuropathy.

What is claimed is:

1. A tricyclic compound of Formula I: 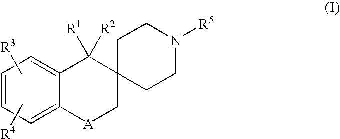
wherein:
R¹ is hydrogen or hydroxy;
R² is hydrogen or hydroxy; or
R¹ and R² together are oxygen;
A is a bond, CH₂, CH CH₃, CH₂ CH₂ or C(CH₃)₂;
R³ and R⁴ are the same or different and are hydrogen, halo, C₁-C₆ alkyl, C₁-C₄ alkoxy, trifluoromethyl, NO₂, COR⁶, COOR⁶ or NR⁶R⁷, wherein R⁶ and R⁷ are the same or different and are hydrogen, C₁-C₆ alkyl or benzyl;
R⁵ is hydrogen, C₁₋₆ alkyl, C₂₋₆ alkenyl, C₃₋₆ cycloalkyl, (O═C)—C₁₋₆alkyl, (O═C)—C₂₋₆ alkenyl, (O═C)—C₃₋₆ cycloalkyl, wherein said alkyl, alkenyl and cycloalkyl groups can be substituted by 1. 2 or 3 groups selected from halo, C₃-C₆ cycloalkyl, phenyl or substituted phenyl, and a pharmaceutically acceptable salt thereof.

2. A compound according to claim 1. wherein R⁵ is C₁-C₆ alkyl, optionally substituted with phenyl or a C₃-C₆ cycloalkyl group.

3. A compound according to claim 1 or 2. wherein R³ is hydrogen, halogen or C₁₋₄ alkoxy.

4. A compound according to any one of claims 1 to 3. wherein R¹ is C₁₋₆ alkyl, C₂₋₆ alkenyl or C₃₋₆ cycloalkyl, optionally substituted by 1. 2 or 3 groups selected from halo, C₃-C₆ cycloalkyl, phenyl or substituted phenyl, and R² is hydrogen.

5. A compound according to any one of claims 1 to 4, wherein R⁴ is hydrogen.

6. A compound selected from the group consisting of:
3.4-Dihydro-1-oxospiro[naphthalene-2(1H),4′-piperidine];
1′-cyclopropylmethyl-3.4-Dihydro-1-oxospiro[naphthalene-2(1H),4′-piperidine];
1′-cyclobutylmethyl-3.4-Dihydro-1-oxospiro[naphthalene-2(1H),4′-piperidine];
1′-cyclohexylmethyl-3.4-Dihydro-1-oxospiro[naphthalene-2(1H),4′-piperidine];
1′-phenylethyl-3.4-Dihydro-1-oxospiro[naphthalene-2(1H),4′-piperidine];
1′-cyclopropylethyl-3.4-Dihydro-1-oxospiro[naphthalene-2(1H),4′-piperidine];
1′-cinnamyl-3.4-Dihydro-1-oxospiro[naphthalene-2(1H),4′-piperidine];
1′-(3.3-diphenylpropyl)-3.4-Dihydro-1-oxospiro[naphthalene-2(1H),4′-piperidine];
1′-(cyclopropylmethyl)-3.4-Dihydro-5.7-dimethyl-1-oxospiro[naphthalene-2(1H),4′-piperidine];
1′-(cyclopropylmethyl)-3.4-Dihydro-6-methoxy-1-oxospiro[naphthalene-2(1H),4′-piperidine];
1′-(cyclopropylmethyl)-3.4-Dihydro-5-methoxy-1-oxospiro[naphthalene-2(1H),4′-piperidine];
1′-(cyclopropylmethyl)-3.4-Dihydro-7-methoxy-1-oxospiro[naphthalene-2(1H),4′-piperidine];
1′-(cyclopropylmethyl)-3.4-Dihydro-7-nitro-1-oxospiro[naphthalene-2(1H),4′-piperidine];
1′-(cyclopropylmethyl)-7-Amino-3.4-dihydro-1-oxospiro[naphthalene-2(1H),4′-piperidine];
1′-(cyclopropylmethyl)-7-Chloro-3.4-dihydro-1-oxospiro[naphthalene-2(1H),4′-piperidine];
3.4-dihydro-1′-methyl-1-oxospiro(naphthalene-2(1H),4′-piperidine);
1′-allyl-3.4-dihydro-1-oxospiro(naphthalene-2(1H),4′-piperidine);
3.4-dihydro-1′-(2-methylpropyl)-1-oxospiro(naphthalene-2(1H),4′-piperidine);
1′-cyclopropionyl-3.4-dihydro-1-oxospiro(naphthalene-2(1H),4′-piperidine);
3.4-dihydro-1-oxospiro(naphthalene-2(1H),4′-piperidine)-1′(trans-2-phenyl-methylcyclopropyl);
3.4-dihydro-1′-benzyl-1-oxospiro(naphthalene-2(1H),4′-piperidine);
3.4-dihydro-1′-(di-p-fluorobenzhydryl)-1-oxospiro(naphthalene-2(1H),4′-piperidine);
1′-cyclopropylmethyl-1.3.4-trihydro-1-hydroxy-spiro(naphthalene-2(1H),4′-piperidine);
1′-cyclopropylmethyl-1.2.3.4-tetrahydro-spiro(naphthalene-2(1H),4′-piperidine);
1′-cyclopropylmethyl-1.3-dihydro-1-oxospiro-[2H-indene-2.4′-piperidine] 1′-(cyclopropylmethyl)-8,9-dihydrospiro[6H-benzocycloheptene-6.4′-piperidin]-5(7H)-one;
1′-cyclopropylmethyl-3.4-dihydro-4-methyl-1-oxospiro(naphthalene-2(1H),4′-piperidine);
6-chloro-1′-cyclopropylmethyl-3.4-dihydro-1-oxospiro(naphthalene-2(1H),4′-piperidine);
1′-cyclopropylmethyl-3.4-dihydro-6-fluoro-1-oxospiro(naphthalene-2(1H),4′-piperidine);
1′-cyclopropylmethyl-3.4-dihydro-6.7-dimethoxy-1-oxospiro(naphthalene-2(1H),4′-piperidine);
1′-(1-cyclopropyl-1-ethyl)-3.4-dihydro-1-oxospiro(naphthalene-2(1H),4′-piperidine);
1′-(3-pentene)-3.4-dihydro-1-oxospiro(naphthalene-2(1H),4′-piperidine;
1′-(3-phenylpropyl)-3.4-dihydro-1-oxospiro(naphthalene-2(1H),4′-piperidine;
1′-cinnamyl-3.4-dihydro-6-methoxy-1-oxospiro(naphthalene-2(1H),4′-piperidine;
1′-cyclopropylethyl-3.4-dihydro-5-methoxy-1-oxospiro(naphthalene-2(1H),4′-piperidine;
1′-cyclobutylmethyl-3.4-dihydro-5-methoxy-1-oxospiro(naphthalene-2(1H),4′-piperidine;
1′-cyclohexylmethyl-3.4-dihydro-5-methoxy-1-oxospiro(naphthalene-2(1H),4′-piperidine;
1′-cinnamyl-3.4-dihydro-5-methoxy-1-oxospiro(naphthalene-2(1H),4′-piperidine;
1′-(2-phenylethyl)-3.4-dihydro-5-methoxy-1-oxospiro(naphthalene-2(1H),4′-piperidine;
1′-(3-phenylpropyl)-3.4-dihydro-5-methoxy-1-oxospiro(naphthalene-2(1H),4′-piperidine;
1′-(3.3′diphenylpropyl)-3.4-dihydro-5-methoxy-1-oxospiro(naphthalene-2(1H),4′-piperidine;
1′-cyclopropylethyl-3.4-dihydro-6-methoxy-1-oxospiro(naphthalene-2(1H),4′-piperidine;
1′-cyclobutylmethyl-3.4-dihydro-6-methoxy-1-oxospiro(naphthalene-2(1H),4′-piperidine;
1′-cyclohexylmethyl-3.4-dihydro-6-methoxy-1-oxospiro(naphthalene-2(1H),4′-piperidine;
1′-cinnamyl-3.4-dihydro-6-methoxy-1-oxospiro(naphthalene-2(1H),4′-piperidine;
1′-(2-phenylethyl)-3.4-dihydro-6-methoxy-1-oxospiro(naphthalene-2(1H),4′-piperidine;
1′-(3-phenylpropyl)-3.4-dihydro-6-methoxy-1-oxospiro(naphthalene-2(1H),4′-piperidine;
1′-(3.3′diphenylpropyl)-3.4-dihydro-6-methoxy-1-oxospiro(naphthalene-2(1H),4′-piperidine;
1′-cyclopropylethyl-3.4-dihydro-7-methoxy-1-oxospiro(naphthalene-2(1H),4′-piperidine;
1′-cyclobutylmethyl-3.4-dihydro-7-methoxy-1-oxospiro(naphthalene-2(1H),4′-piperidine;
1′-cyclohexylmethyl-3.4-dihydro-7-methoxy-1-oxospiro(naphthalene-2(1H),4′-piperidine;
1′-cinnamyl-3.4-dihydro-7-methoxy-1-oxospiro(naphthalene-2(1H),4′-piperidine;
1′-(2-phenylethyl)-3.4-dihydro-7-methoxy-1-oxospiro(naphthalene-2(1H),4′-piperidine;
1′-(3-phenylpropyl)-3.4-dihydro-7-methoxy-1-oxospiro(naphthalene-2(1H),4′-piperidine;
1′-(3.3′diphenylpropyl)-3.4-dihydro-7-methoxy-1-oxospiro(naphthalene-2(1H),4′-piperidine;
1′-cyclopropylethyl-3.4-dihydro-4-methyl-1-oxospiro(naphthalene-2(1H),4′-piperidine;
1′-cyclobutylmethyl-3.4-dihydro-4-methyl-1-oxospiro(naphthalene-2(1H),4′-piperidine;
1′-cyclohexylmethyl-3.4-dihydro-4-methyl-1-oxospiro(naphthalene-2(1H),4′-piperidine;
1′-cinnamyl-3.4-dihydro-4-methyl-1-oxospiro(naphthalene-2(1H),4′-piperidine;
1′-(2-phenylethyl)-3.4-dihydro-4-methyl-1-oxospiro(naphthalene-2(1H),4′-piperidine;
1′-(3-phenylpropyl)-3.4-dihydro-4-methyl-1-oxospiro(naphthalene-2(1H),4′-piperidine;
1′-(3.3′diphenylpropyl)-3.4-dihydro-4-methyl-1-oxospiro(naphthalene-2(1H),4′-piperidine;
6-chloro-1′-cyclopropylethyl-3.4-dihydro-1-oxospiro(naphthalene-2(1H),4′-piperidine;
6-chloro-1′-cyclobutylmethyl-3.4-dihydro-1-oxospiro(naphthalene-2(1H),4′-piperidine;
6-chloro-1′-cyclohexylmethyl-3.4-dihydro-1-oxospiro(naphthalene-2(1H),4′-piperidine;
6-chloro-1′-cinnamyl-3.4-dihydro-1-oxospiro(naphthalene-2(1H),4′-piperidine;
6-chloro-1′-(2-phenylethyl)-3.4-dihydro-1-oxospiro(naphthalene-2(1H),4′-piperidine;
6-chloro-1′-(3-phenylpropyl)-3.4-dihydro-1-oxospiro(naphthalene-2(1H),4′-piperidine; and
6-chloro-1′-(3.3′diphenylpropyl)-3.4-dihydro-1-oxospiro(naphthalene-2(1H),4′-piperidine.
3.4-Dihydro-6-methoxy-1-oxospiro[naphthalene-2(1H),4′-piperidine]

7. A compound selected from the group consisting of:
1′-cyclopropylmethyl-3.4-Dihydro-1-oxospiro[naphthalene-2(1H),4′-piperidine];
1′-cyclopropylethyl-3.4-Dihydro-1-oxospiro[naphthalene-2(1H),4′-piperidine];
1′-cinnamyl-3.4-Dihydro-1-oxospiro[naphthalene-2(1H),4′-piperidine];
1′-(3.3-diphenylpropyl)-3.4-Dihydro-1-oxospiro[naphthalene-2(1H),4′-piperidine];
1′-(cyclopropylmethyl)-3.4-Dihydro-5.7-dimethyl-1-oxospiro[naphthalene-2(1H),4′-piperidine];
1′-(cyclopropylmethyl)-3.4-Dihydro-6-methoxy-1-oxospiro[naphthalene-2(1H),4′-piperidine];
1′-(cyclopropylmethyl)-3.4-Dihydro-5-methoxy-1-oxospiro[naphthalene-2(1H),4′-piperidine];
1′-(cyclopropylmethyl)-3.4-Dihydro-7-methoxy-1-oxospiro[naphthalene-2(1H),4′-piperidine];
1′-(cyclopropylmethyl)-3.4-Dihydro-7-nitro-1-oxospiro[naphthalene-2(1H),4′-piperidine];
1′-(cyclopropylmethyl)-7-Amino-3.4-dihydro-1-oxospiro[naphthalene-2(1H),4′-piperidine];
1′-(cyclopropylmethyl)-7-Chloro-3.4-dihydro-1-oxospiro[naphthalene-2(1H),4′-piperidine];
1′-cyclopropylmethyl-1.3.4-trihydro-1-hydroxy-spiro(naphthalene-2(1H),4′-piperidine);
1′-cyclopropylmethyl-1.2.3.4-tetrahydro-spiro(naphthalene-2(1H),4′-piperidine);
1′-cyclopropylmethyl-1.3-dihydro-1-oxospiro-[2H-indene-2.4′-piperidine]
1′-(cyclopropylmethyl)-8,9-dihydrospiro[6H-benzocycloheptene-6.4′-piperidin]-5(7H)-one;
1′-cyclopropylmethyl-3.4-dihydro-4-methyl-1-oxospiro(naphthalene-2(1H),4′-piperidine);
6-chloro-1′-cyclopropylmethyl-3.4-dihydro-1-oxospiro(naphthalene-2(1H),4′-piperidine);
1′-cyclopropylmethyl-3.4-dihydro-6-fluoro-1-oxospiro(naphthalene-2(1H),4′-piperidine);
1′-cyclopropylmethyl-3.4-dihydro-6.7-dimethoxy-1-oxospiro(naphthalene-2(1H),4′-piperidine);
1′-(1-cyclopropyl-1-ethyl)-3.4-dihydro-1-oxospiro(naphthalene-2(1H),4′-piperidine);
1′-(3-pentene)-3.4-dihydro-1-oxospiro(naphthalene-2(1H),4′-piperidine;
1′-(3-phenylpropyl)-3.4-dihydro-1-oxospiro(naphthalene-2(1H),4′-piperidine;
1′-cinnamyl-3.4-dihydro-6-methoxy-1-oxospiro(naphthalene-2(1H),4′-piperidine;
1′-cyclopropylethyl-3.4-dihydro-5-methoxy-1-oxospiro(naphthalene-2(1H),4′-piperidine;
1′-cyclobutylmethyl-3.4-dihydro-5-methoxy-1-oxospiro(naphthalene-2(1H),4′-piperidine;
1′-cinnamyl-3.4-dihydro-5-methoxy-1-oxospiro(naphthalene-2(1H),4′-20 piperidine;
1′-(3-phenylpropyl)-3.4-dihydro-5-methoxy-1-oxospiro(naphthalene-2(1H),4′-piperidine;
1′-(3.3′diphenylpropyl)-3.4-dihydro-5-methoxy-1-oxospiro(naphthalene-2(1H),4′-piperidine;
1′-cyclopropylethyl-3.4-dihydro-6-methoxy-1-oxospiro(naphthalene-2(1H),4′-piperidine;
1′-cyclobutylmethyl-3.4-dihydro-6-methoxy-1-oxospiro(naphthalene-2(1H),4′-piperidine;
1′-cinnamyl-3.4-dihydro-6-methoxy-1-oxospiro(naphthalene-2(1H),4′-30 piperidine;
1′-(3-phenylpropyl)-3.4-dihydro-6-methoxy-1-oxospiro(naphthalene-2(1H),4′-piperidine;
1′-(3.3′diphenylpropyl)-3.4-dihydro-6-methoxy-1-oxospiro(naphthalene-2(1H),4′-piperidine;
1′-cyclopropylethyl-3.4-dihydro-7-methoxy-1-oxospiro(naphthalene-2(1H),4′-piperidine;
1′-cinnamyl-3.4-dihydro-7-methoxy-1-oxospiro(naphthalene-2(1H),4′-piperidine;
1′-(3-phenylpropyl)-3.4-dihydro-7-methoxy-1-oxospiro(naphthalene-2(1H),4′-piperidine;
1′-(3.3′diphenylpropyl)-3.4-dihydro-7-methoxy-1-oxospiro(naphthalene-2(1H),4′-piperidine;
1′-cyclopropylethyl-3.4-dihydro-4-methyl-1-oxospiro(naphthalene-2(1H),4′-piperidine;
1′-cyclobutylmethyl-3.4-dihydro-4-methyl-1-oxospiro(naphthalene-2(1H),4′-piperidine;
1′-cinnamyl-3.4-dihydro-4-methyl-1-oxospiro(naphthalene-2(1H),4′-piperidine;
1′-(3-phenylpropyl)-3.4-dihydro-4-methyl-1-oxospiro(naphthalene-2(1H),4′-piperidine;
1′-(3.3′diphenylpropyl)-3.4-dihydro-4-methyl-1-oxospiro(naphthalene-2(1H),4′-piperidine;
6-chloro-1′-cyclopropylethyl-3.4-dihydro-1-oxospiro(naphthalene-2(1H),4′-piperidine;
6-chloro-1′-cinnamyl-3.4-dihydro-1-oxospiro(naphthalene-2(1H),4′-piperidine;
6-chloro-1′-(3-phenylpropyl)-3.4-dihydro-1-oxospiro(naphthalene-2(1H),4′-piperidine; and
6-chloro-1′-(3.3′diphenylpropyl)-3.4-dihydro-1-oxospiro(naphthalene-2(1H),4′-piperidine.
3.4-Dihydro-6-methoxy-1-oxospiro[naphthalene-2(1H),4′-piperidine]

8. A compound selected from the group consisting of:
1′-cyclopropylmethyl-3.4-Dihydro-1-oxospiro[naphthalene-2(1H),4′-piperidine];
1′-cyclopropylethyl-3.4-Dihydro-1-oxospiro[naphthalene-2(1H),4′-piperidine];
1′-cinnamyl-3.4-Dihydro-1-oxospiro[naphthalene-2(1H),4′-piperidine];
1′-(3.3-diphenylpropyl)-3.4-Dihydro-1-oxospiro[naphthalene-2(1H),4′-piperidine];
1′-(cyclopropylmethyl)-3.4-Dihydro-6-methoxy-1-oxospiro[naphthalene-2(1H),4′-piperidine];
1′-(3-phenylpropyl)-3.4-dihydro-1-oxospiro(naphthalene-2(1H),4′-piperidine;
1′-cyclobutylmethyl-3.4-dihydro-5-methoxy-1-oxospiro(naphthalene-2(1H),4′-piperidine;
1′-cyclopropylethyl-3.4-dihydro-6-methoxy-1-oxospiro(naphthalene-2(1H),4′-piperidine;
1′-cyclobutylmethyl-3.4-dihydro-6-methoxy-1-oxospiro(naphthalene-2(1H),4′-piperidine;
1′-(3-phenylpropyl)-3.4-dihydro-6-methoxy-1-oxospiro(naphthalene-2(1H),4′-piperidine;
1′-cyclopropylethyl-3.4-dihydro-7-methoxy-1-oxospiro(naphthalene-2(1H),4′-piperidine;
3.4-Dihydro-6-methoxy-1-oxospiro [naphthalene-2(1H),4′-piperidine]

9. A pharmaceutical composition comprising a compound according to any one of claims 1 to 8 admixed with a pharmaceutically acceptable carrier, diluent, or carrier therefor.

10. A method for treating a mammal suffering from pain and in need of treatment comprising administering an effective amount of a compound of any one of claims 1 to 8.

11. A method according to claim 10 wherein the pain is neuropathic pain.

12. A method according to claim 10 wherein the pain is diabetic neuropathy.

13. A method for treating a mammal suffering from a seizure disorder comprising administering an effective amount of a compound of any one of claims 1 to 8.
